(19) United States
(12) Patent Application Publication
(10) Pub. No.: US 2019/0322625 A1
(43) Pub. Date: Oct. 24, 2019

(54) MONO-(ACID) SALTS OF 6-AMINOISOQUINOLINES AND USES THEREOF

(71) Applicant: Aerie Pharmaceuticals, Inc., Bedminster, NJ (US)

(72) Inventors: Mitchell A. deLong, Chapel Hill, NC (US); Jill M. Sturdivant, Chapel Hill, NC (US); Cynthia L. Lichorowic, Raleigh, NC (US); Cheng-Wen Lin, Cary, NC (US)

(21) Appl. No.: 16/370,240

(22) Filed: Mar. 29, 2019

Related U.S. Application Data
(60) Provisional application No. 62/650,687, filed on Mar. 30, 2018.

(51) Int. Cl. C07D 217/04; A61P 27/02; A61P 27/06; A61K 9/00; A61P 29/00; C07C 309/30

(52) U.S. Cl. CPC C07D 217/04; A61P 27/02; A61P 27/06; C07B 2200/13; A61P 29/00; C07C 309/30; A61K 9/0048

(57) ABSTRACT

Provided herein are mono-(acid) salts of a compound of a Formulae provided herein. Also provided herein are compositions comprising a mono-(acid) salt of a compound of a Formulae provided herein. Also provided herein are pharmaceutical compositions comprising a mono-(acid) salt of a compound of a Formulae provided herein. Also provided herein are methods of treating a disease in a subject in need thereof, comprising administering to the subject a therapeutically effective amount of a mono-(acid) salt of a compound of a Formulae provided herein.

### RELATED APPLICATIONS

This application claims the priority of U.S. Provisional Patent Application No. 62/650,687, filed Mar. 30, 2018, the entire content of which is incorporated herein by reference.

### INTRODUCTION

A variety of hormones, neurotransmitters and biologically active substances control, regulate or adjust the functions of living bodies via specific receptors located in cell membranes. Many of these receptors mediate the transmission of intracellular signals by activating guanine nucleotide-binding proteins (G proteins) to which the receptor is coupled. Such receptors are generically referred to as G-protein coupled receptors (GPCRs) and include, among others, α-adrenergic receptors, β-adrenergic receptors, opioid receptors, cannabinoid receptors and prostaglandin receptors. The biological effects of activating these receptors is not direct but is mediated by a ‘downstream’ host of intracellular proteins. One class of these downstream effectors is the “kinase” class.

The various kinases play roles in the regulation of various physiological functions. For example, kinases have been implicated in a number of disease or disorder states, including, but not limited to: cardiac indications such as angina pectoris, essential hypertension, myocardial infarction, supraventricular and ventricular arrhythmias, congestive heart failure, atherosclerosis, renal failure, diabetes, respiratory indications such as asthma, chronic bronchitis, bronchospasm, emphysema, airway obstruction, upper respiratory indications such as rhinitis, seasonal allergies, inflammatory disease, inflammation in response to injury, rheumatoid arthritis. Other diseases or disorders include chronic inflammatory bowel disease, glaucoma, hypergastrinemia, gastrointestinal indications such as acid/peptic disorder, erosive esophagitis, gastrointestinal hypersecretion, mastocytosis, gastrointestinal reflux, peptic ulcer, Zollinger-Ellison syndrome, pain, obesity, bulimia nervosa, depression, obsessive-compulsive disorder, organ malformations (e.g., cardiac malformations), neurodegenerative diseases such as Parkinson's Disease and Alzheimer's Disease, multiple sclerosis, Epstein-Barr infection and cancer. In other disease states, the role of kinases is only now becoming clear. The retina is a complex tissue composed of multiple interconnected cell layers, highly specialized for transforming light and color into electrical signals that are perceived by the brain. Damage or death of the primary light-sensing cells, the photoreceptors, results in devastating effects on vision. Despite the identification of numerous mutations that cause inherited retinal degenerations, the cellular and molecular mechanisms leading from the primary mutations to photoreceptor apoptosis are not well understood.

The balance between the initiation and the inactivation of intracellular signals determines the intensity and duration of the response of the receptors to stimuli such as agonists. When desensitization occurs, the mediation or regulation of the physiological function mediated or regulated by the G proteins to which the receptors are coupled is reduced or prevented. For example, when agonists are administered to treat a disease or disorder by activation of certain receptors, the receptors relatively quickly become desensitized from the action of the GRKs such that agonist administration may no longer result in therapeutic activation of the appropriate receptors. At that point, administration of the agonist no longer enables sufficient or effective control of or influence on the disease or disorder intended to be treated.

In view of the role that kinases have in many disease states, there is an urgent and continuing need for small molecule ligands which inhibit or modulate the activity of kinases. Without wishing to be bound by theory, it is thought that modulation of the activity of kinases by the mono-(acid) salts of the present disclosure is responsible for their beneficial effects.

Additionally, a number of ocular disorders are caused or aggravated by damage to the optic nerve head, degeneration of ocular tissues, or elevated intraocular pressure (IOP). For example, “glaucomas” are a group of debilitating eye diseases that are a leading cause of irreversible blindness in the United States and other developed nations. There is a continuing need for therapies that control elevated IOP to limit glaucomatous damage without undesirable side-effects.

In addition, compounds dosed directly into the back of the eye must be in a form that prevents their too rapid dispersal, in a form that allows for ease of injection, and compatible with the excipients that are post of the dosage form The drug product, if composed of a small organic molecule encased in a biodegradable polymer matrix, must be compatible for months in an aqueous environment with a pH of approximately 7.4 and a temperature of 37 deg. C., without swelling or deforming to ensure continuous delivery of the active agent.

While compounds provided herein can be made as free bases, mono-(acid) salts, or di salts, it has been unexpectedly found that the mono-(acid) salts, particularly the mono-(acid) salts of non-nucleophilic, organic strong acids, possess excellent stability under the above described conditions, and are thus suitable for treatment of eye diseases and other diseases wherein the drug is to be dosed in a polymer matrix inside of a human or animal for a period of weeks to years.

### SUMMARY

In one aspect, provided herein are mono-(acid) salts of a compound of Formula (I):

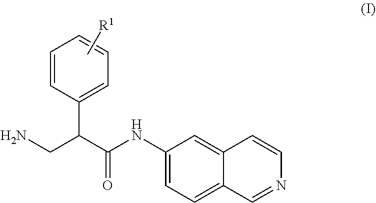

wherein
R¹ is H, halogen, —OH, —C₁₋₆ alkyl, —C₁₋₆ alkyl(R²)ₘ, —(CH₂)ₙOC(O)—(C₁₋₆ alkyl(R³)ₚ), or —(CH₂)ₙOC(O)—(C₆₋₁₀ aryl(R³)ₚ);
each R² is, independently, —OH or halogen;
each R³ is, independently, hydrogen, —OH, halogen, —C₁₋₆ alkyl, mono-halogen C₁₋₆ alkyl, di-halogen C₁₋₆ alkyl, or tri-halogen C₁₋₆ alkyl;
m is 1, 2, 3, 4, 5, or 6;
n is 1, 2, or 3; and
p is 0, 1, 2, 3, 4, 5, or 6.

In another aspect, provided herein are compositions comprising a mono-(acid) salt of Formula (I).

In another aspect, provided herein are pharmaceutical compositions comprising a mono-(acid) salt of Formula (I), and a pharmaceutically acceptable carrier.

In another aspect, provided herein are methods of treating a disease in a subject in need thereof, comprising administering to the subject a therapeutically effective amount of a mono-(acid) salt of Formula (I).

In another aspect, provided herein are methods of reducing intraocular pressure in a subject in need thereof, comprising administering to the subject a therapeutically effective amount of a mono-(acid) salt of Formula (I).

In another aspect, provided herein are methods of inhibiting a kinase in a cell, comprising contacting the cell with an amount of a mono-(acid) salt of Formula (I) effective to inhibit the kinase.

In another aspect, provided herein are methods of treating back of the eye diseases such as DME, wet and dry AMD, uveitis, glaucoma, and diabetic retinopathy, by dosing these mono-(acid) salts in an appropriate polymer matrix to the back of the eye.

In another aspect, provided herein are kits comprising a mono-(acid) salt of Formula (I) and instructions for use thereof.

In another aspect, provided herein are articles of manufacture comprising a mono-(acid) salt of Formula (I) and instructions for use thereof.

### BRIEF DESCRIPTION OF THE DRAWINGS

FIG. 1. is a comparison of an X-ray powder diffraction (XRPD) pattern of di-(hydrochloric acid) salt of (S)-4-(3-amino-1-(isoquinolin-6-ylamino)-1-oxopropan-2-yl)benzyl 2,4-dimethylbenzoate (File: 786391.sum trace) with an XRPD pattern of mono-(hydrochloric acid) salt of (S)-4-(3-amino-1-(isoquinolin-6-ylamino)-1-oxopropan-2-yl)benzyl 2,4-dimethylbenzoate (File: 780626.sum trace).

### DETAILED DESCRIPTION

Provided herein are mono-(acid) salts of the compounds of the Formulae provided herein, compositions comprising the mono-(acid) salts, and pharmaceutical compositions comprising the mono-(acid) salts, which are useful in the treatment and prevention of disease (e.g., diseases associated with Rho kinase activity, e.g. ocular disorders).

### Definitions

Listed below are definitions of various terms used to describe the present disclosure. These definitions apply to the terms as they are used throughout this specification and claims, unless otherwise limited in specific instances, either individually or as part of a larger group.

Unless defined otherwise, all technical and scientific terms used herein generally have the same meaning as commonly understood by one of ordinary skill in the art to which the present disclosure belongs. Generally, the nomenclature used herein and the laboratory procedures in cell culture, molecular genetics, organic chemistry, and peptide chemistry are those well-known and commonly employed in the art.

Publications and patents are referred to throughout this disclosure. All U.S. patents cited herein are hereby incorporated by reference. All percentages, ratios, and proportions used herein are percent by weight unless otherwise specified.

As used herein, the articles “a” and “an” refer to one or to more than one (i.e. to at least one) of the grammatical object of the article. By way of example, “an element” means one element or more than one element. Furthermore, use of the term “including” as well as other forms, such as “include”, “includes,” and “included,” is not limiting.

As used herein, the term “about” will be understood by persons of ordinary skill in the art and will vary to some extent on the context in which it is used. As used herein when referring to a measurable value such as an amount, a temporal duration, and the like, the term “about” is meant to encompass variations of ±20% or ±10%, including ±5%, ±1%, and ±0.1% from the specified value, as such variations are appropriate to perform the disclosed methods.

As used herein, the term “alkyl,” by itself or as part of another substituent means, unless otherwise stated, a straight or branched chain hydrocarbon having the number of carbon atoms designated (i.e., C₁₋₆-alkyl means one to six carbon atoms) and includes straight and branched chains. Examples include methyl, ethyl, n-propyl, isopropyl, n-butyl, isobutyl, tert-butyl, pentyl, neopentyl, hexyl, and the like.

As used herein, the term “aromatic” refers to a carbocycle with one or more polyunsaturated rings and having aromatic character, i.e., having (4n+2) delocalized π (pi) electrons, where n is an integer.

As used herein, the term “aryl,” employed alone or in combination with other terms, means, unless otherwise stated, a carbocyclic aromatic system containing one or more rings (typically one, two, or three rings), wherein such rings may be attached together in a pendent manner, such as a biphenyl, or may be fused, such as naphthalene. Examples of aryl groups include phenyl, anthracyl, and naphthyl. Some examples are phenyl (e.g., C₆-aryl) and biphenyl (e.g., C₁₂-aryl). In some embodiments, aryl groups have from six to sixteen carbon atoms. In some embodiments, aryl groups have from six to twelve carbon atoms (e.g., C₆₋₁₂-aryl). In some embodiments, aryl groups have six carbon atoms (e.g., C₆-aryl).

As used herein, the term “biodegradable polymeric matrix” refers to a polymeric matrix that degrades in a biological system, either with the aid of the system's naturally-occurring enzymes, or independent of the action of enzymes.

As used herein, the term “composition” or “pharmaceutical composition” refers to a mixture of at least one mono-(acid) salt, i.e., a mono-(acid) salt of a compound of the Formulae provided herein, useful as described herein with a pharmaceutically acceptable carrier. The pharmaceutical composition facilitates administration of the mono-(acid) salt to a patient or subject. Multiple techniques of administering a mono-(acid) salt exist in the art including, but not limited to, intravenous, oral, aerosol, parenteral, ophthalmic, pulmonary, and topical administration.

As used herein, the term “contacting a cell” is used to mean contacting a cell in vitro or in vivo (i.e. in a subject, such as a mammal, including humans, cats and dogs).

As used herein, the term “controlling the disease or disorder” is used to mean changing the activity of one or more kinases to affect the disease or disorder.

As used herein, the term “disease or disorder associated with kinase activity” is used to mean a disease or disorder treatable, in whole or in part, by inhibition of one or more kinases.

As used herein, the terms “effective amount,” “pharmaceutically effective amount” and “therapeutically effective amount” refer to a nontoxic but sufficient amount of an agent to provide the desired biological result. That result may be reduction or alleviation of the signs, symptoms, or causes of a disease, or any other desired alteration of a biological system. An appropriate therapeutic amount in any individual case may be determined by one of ordinary skill in the art using routine experimentation.

As used herein, the term “eye disease” or “ocular disorder” includes, but is not limited to, glaucoma, allergy, inflammatory eye disease, ocular hypertension, cancers of the eye, neurodegenerative diseases of the eye such as diabetic macular edema (DME) and wet or dry age-related macular degeneration (AMD), uveitis, diabetic retinopathy, and dry eye.

As used herein, the term “halo” or “halogen” alone or as part of another substituent means, unless otherwise stated, a fluorine, chlorine, bromine, or iodine atom, e.g., fluorine, chlorine, or bromine, e.g., fluorine or chlorine.

As used herein, the term “haloalkyl” refers to alkyl radicals wherein any one or more of the alkyl carbon atoms is substituted with halo as defined above. Haloalkyl embraces monohaloalkyl, dihaloalkyl, and polyhaloalkyl radicals. The term “haloalkyl” includes, but is not limited to, fluoromethyl, difluoromethyl, trifluoromethyl, chloromethyl, dichloromethyl, trichloromethyl, and pentafluoroethyl.

As used herein, the term “mono-(acid) salt” refers to an anion-cation pair formed from the combination of 1) a single acid (e.g., a single molecule that—when ionized upon combination with a compound of one of the Formulae provided herein—forms a monoatomic or polyatomic anion), and 2) a single compound of one of the Formulae provided herein (e.g., a single compound of one of the Formulae provided herein that—when ionized upon combination with an acid—forms a cation of a compound of one of the Formulae provided herein). Thus, mono-(acid) salts provided herein may be referred to as a mono-(acid) salt of a compound of one of the Formulae provided herein.

As used herein, the term “patient,” “individual” or “subject” refers to a human or a non-human mammal. Non-human mammals include, for example, livestock and pets, such as ovine, bovine, porcine, canine, feline and murine mammals. In some embodiments, the patient, subject, or individual is human.

As used herein, the term “pharmaceutically acceptable” refers to a material, such as a carrier or diluent, which does not abrogate the biological activity or properties of the mono-(acid) salt, and is relatively non-toxic, i.e., the material may be administered to an individual without causing undesirable biological effects or interacting in a deleterious manner with any of the components of the composition in which it is contained.

As used herein, the term “pharmaceutically acceptable carrier” means a pharmaceutically acceptable material, composition or carrier, such as a liquid or solid filler, stabilizer, dispersing agent, suspending agent, diluent, excipient, thickening agent, solvent or encapsulating material, involved in carrying or transporting a mono-(acid) salt useful as described herein within or to the patient such that it may perform its intended function. Typically, such constructs are carried or transported from one organ, or portion of the body, to another organ, or portion of the body. Each carrier must be “acceptable” in the sense of being compatible with the other ingredients of the formulation, including the mono-(acid) salt useful as described herein, and not injurious to the patient. As used herein, “pharmaceutically acceptable carrier” also includes any and all coatings, antibacterial and antifungal agents, and absorption delaying agents, and the like that are compatible with the activity of the mono-(acid) salt useful as described herein, and are physiologically acceptable to the patient. Other additional ingredients that may be included in the pharmaceutical compositions used in the practice of the present disclosure are described, for example in Remington's Pharmaceutical Sciences (Genaro, Ed., Mack Publishing Co., 1985, Easton, Pa.), which is incorporated herein by reference.

As used herein, the term “prevent” or “prevention” means no disorder or disease development if none had occurred, or no further disorder or disease development if there had already been development of the disorder or disease. Also considered is the ability of one to prevent some or all of the symptoms associated with the disorder or disease.

As used herein, “safe and effective amount” means an amount of a mono-(acid) salt sufficient to significantly induce a positive modification in the disease or disorder to be treated, but low enough to avoid serious side effects (at a reasonable benefit/risk ratio), within the scope of sound medical judgment. A safe and effective amount of a mono-(acid) salt will vary with the particular disease or disorder being treated, the age and physical condition of the patient being treated, the severity of the disease or disorder, the duration of treatment, the nature of concurrent therapy, the particular pharmaceutically-acceptable carrier utilized, and like factors within the knowledge and expertise of the attending physician.

As used herein, the term “treatment” or “treating,” is defined as the application or administration of a therapeutic agent, i.e., a mono-(acid) salt provided herein, to a patient, or application or administration of the therapeutic agent to an isolated tissue or cell line from a patient (e.g., for diagnosis or ex vivo applications), who has a disease or disorder treatable by administration of the therapeutic agent, with the purpose to heal, alleviate, relieve, alter, remedy, ameliorate, improve or affect the disease or disorder, the symptoms of the disease or disorder, or the potential to develop the disease or disorder. Such treatments may be specifically tailored or modified, based on knowledge obtained from the field of pharmacogenomics.

### Mono-(Acid) Salts

In one aspect, provided herein are mono-(acid) salts of a compound of Formula (I):

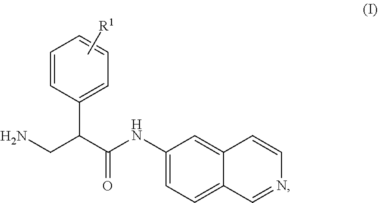

which compounds are useful for the treatment of a disease in a subject in need thereof.

In one aspect, provided herein is a mono-(acid) salt of a compound of Formula (I):

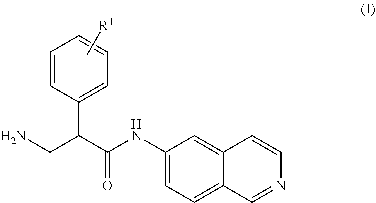

wherein
R¹ is H, halogen, —OH, —C₁₋₆ alkyl, —C₁₋₆ alkyl(R²)ₘ, —(CH₂)ₙOC(O)—(C₁₋₆ alkyl(R³)ₚ), or —(CH₂)ₙOC(O)—(C₆₋₁₀ aryl(R³)_{P});
each R² is, independently, —OH or halogen;
each R³ is, independently, hydrogen, —OH, halogen, —C₁₋₆ alkyl, mono-halogen C₁₋₆ alkyl, di-halogen C₁₋₆ alkyl, or tri-halogen C₁₋₆ alkyl;
m is 1, 2, 3, 4, 5, or 6;
n is 1, 2, or 3; and
p is 0, 1, 2, 3, 4, 5, or 6.

In some embodiments, the mono-(acid) salt is an anion-cation pair formed from a single monoatomic anion and a cation of a compound of Formula (I).

In some embodiments, the mono-(acid) salt is an anion-cation pair formed from a single polyatomic anion and a cation of a compound of Formula (I).

In some embodiments, the acid is (−)-L-malic acid, (−)-L-pyroglutamic acid, (+)-camphor-10-sulfonic acid, (+)-camphoric acid, (+)-L-tartaric acid, 1-hydroxy-2-naphthoic acid, 2,2-dichloro-acetic acid, 2-hydroxy-ethanesulfonic acid, 2-oxo-glutaric acid, 4-acetamido-benzoic acid, 4-amino-salicylic acid, acetic acid, adipic acid, alginic acid, benzenesulfonic acid, benzoic acid, capric acid, caproic acid, caprylic acid, carbonic acid, cinnamic acid, citric acid, cyclamic acid, D-glucoheptonic acid, D-gluconic acid, D-glucuronic acid, DL-lactic acid, DL-mandelic acid, dodecylsulfuric acid, ethane-1,2-disulfonic acid, ethanesulfonic acid, formic acid, fumaric acid, galactaric acid, gentisic acid, glutamic acid, glutaric acid, glycerophosphoric acid, glycolic acid, hippuric acid, hydrobromic acid, hydrochloric acid, isobutyric acid, lactobionic acid, L-ascorbic acid, L-aspartic acid, lauric acid, maleic acid, malonic acid, methanesulfonic acid, naphthalene-1,5-disulfonic acid, naphthalene-2-sulfonic acid, nicotinic acid, nitric acid, oleic acid, orotic acid, oxalic acid, palmitic acid, pamoic acid, phosphoric acid, propionic acid, p-toluenesulfonic acid, salicylic acid, sebacic acid, stearic acid, succinic acid, sulfuric acid, thiocyanic acid, or undecylenic acid.

In some embodiments, the acid is hydrochloric acid, methanesulfonic acid, p-toluenesulfonic acid, benzoic acid, sulfuric acid, acetic acid, phosphoric acid, citric acid, benzenesulfonic acid, sorbic acid, D-aspartic acid, L-aspartic acid, DL-aspartic acid, tartaric acid, fumaric acid, maleic acid, 2,4-dimethylbenzenesulfonic acid, phenylmethanesulfonic acid, 4-chlorobenzenesulfonic acid, [1,1′-biphenyl]-4-sulfonic acid, or cyclopentane sulfonic acid.

In some embodiments, the acid is methanesulfonic acid, p-toluenesulfonic acid, benzenesulfonic acid, 2,4-dimethylbenzenesulfonic acid, phenylmethanesulfonic acid, 4-chlorobenzenesulfonic acid, [1,1′-biphenyl]-4-sulfonic acid, or cyclopentane sulfonic acid.

In some embodiments, the compound of Formula (I) is a compound of Formula (Ia):

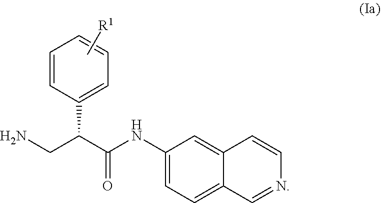

In some embodiments, the compound of Formula (I) is a compound of Formula (Ib):

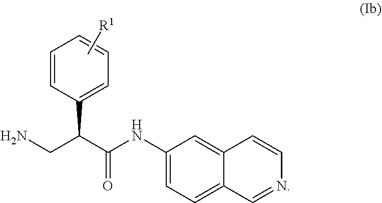

In some embodiments, the compound of Formula (I) is a compound of Formula (II):

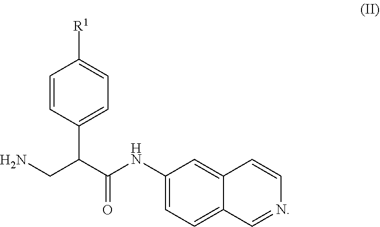

In some embodiments, the compound of Formula (II) is a compound of Formula (IIa):

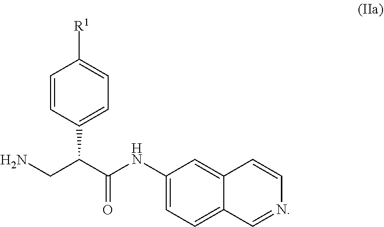

In some embodiments, the compound of Formula (II) is a compound of Formula (IIb):

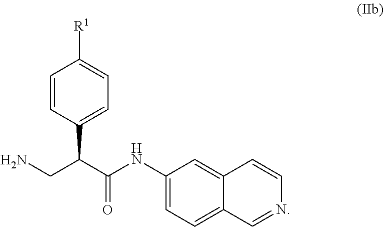

In some embodiments, the compound of Formula (I) is a compound of Formula (III):

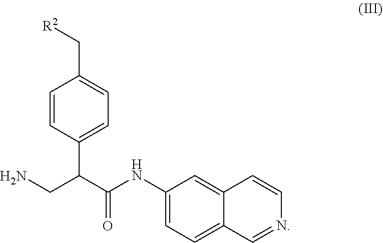

In some embodiments, the compound of Formula (III) is a compound of Formula (IIIa):

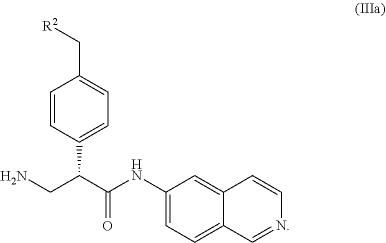

In some embodiments, the compound of Formula (III) is a compound of Formula (IIIb):

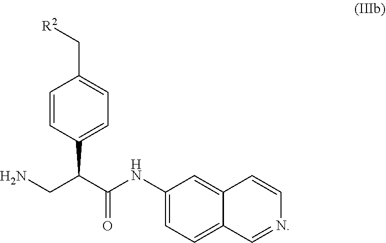

In some embodiments, the compound of Formula (I) is a compound of Formula (IV):

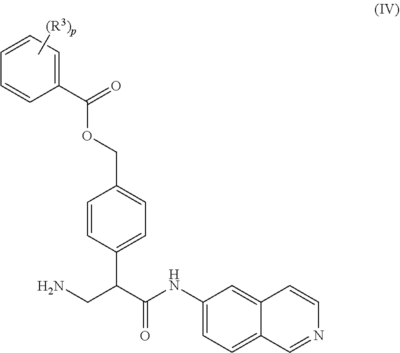

wherein
p is 0, 1, 2, 3, 4, or 5.

In some embodiments, the compound of Formula (IV) is a compound of Formula (IVa):

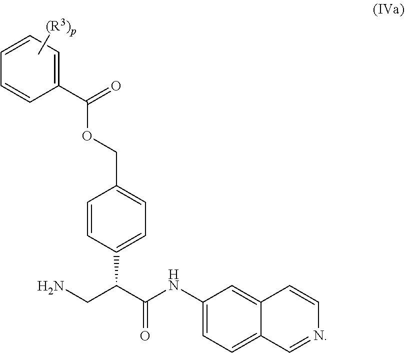

In some embodiments, the compound of Formula (IV) is a compound of Formula (IVb):

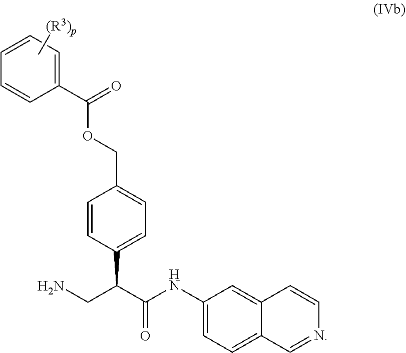

In some embodiments of these Formulae, p is 1, 2, 3, 4 or 5. In some embodiments, p is 0, 1, 2, or 3. In some embodiments, p is 1, 2, or 3. In some embodiments, p is 2.

In some embodiments, R¹ is H, halogen, —OH, —C₁₋₆ alkyl, —C₁₋₆ alkyl(R²)ₘ, or —(CH₂)ₙOC(O)—(C₆₋₁₀ aryl(R³)ₚ). In some embodiments, R¹ is H, halogen, —OH, —C₁₋₆ alkyl, or —C₁₋₆ alkyl(R²)ₘ. In some embodiments, R¹ is halogen, —OH, methyl, ethyl, —C alkyl(R²)ₘ, or —C₂ alkyl(R²)ₘ. In some embodiments, R¹ is —(CH₂)ₙOC(O)—(C₆₋₁₀ aryl(R³)ₚ). In some embodiments, R¹ is —(CH₂)ₙOC(O)—(C₁₋₆ alkyl(R³)ₚ). In some embodiments, R¹ is —(CH₂)ₙOC(O)-(phenyl(R³)ₚ).

In some embodiments, R² is —OH.

In some embodiments of these Formulae, R¹ is —C₁₋₆ alkyl(R²)ₘ; and R² is —OH.

In some embodiments, each R³ is, independently, hydrogen, OH, halogen, or —C₁₋₆ alkyl. In some embodiments, each R³ is, independently, hydrogen, halogen or —C₁₋₂ alkyl. In some embodiments, each R³ is methyl. In some embodiments, R³ is, independently, —OH, halogen, —C₁₋₆ alkyl, mono-halogen C₁₋₆ alkyl, di-halogen C₁₋₆ alkyl, or tri-halogen C₁₋₆ alkyl; In some embodiments, m is 1, 2, 3 or 4. In some embodiments, m is 1 or 2.

In some embodiments, n is 2. In some embodiments, n is 1.

In some embodiments, the compound of Formula (I) is:

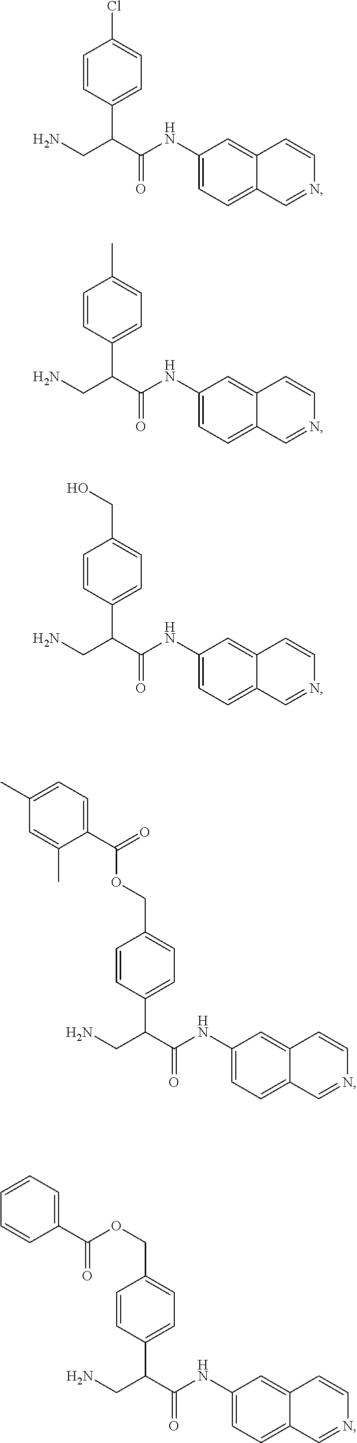 [SMILES: NCC(C(=O)Nc1ccc2cnccc2c1)c1ccc(Cl)cc1 ; Cc1ccc(C(CN)C(=O)Nc2ccc3cnccc3c2)cc1 ; NCC(C(=O)Nc1ccc2cnccc2c1)c1ccc(CO)cc1 ; Cc1ccc(C(=O)OCc2ccc(C(CN)C(=O)Nc3ccc4cnccc4c3)cc2)c(C)c1 ; NCC(C(=O)Nc1ccc2cnccc2c1)c1ccc(COC(=O)c2ccccc2)cc1]

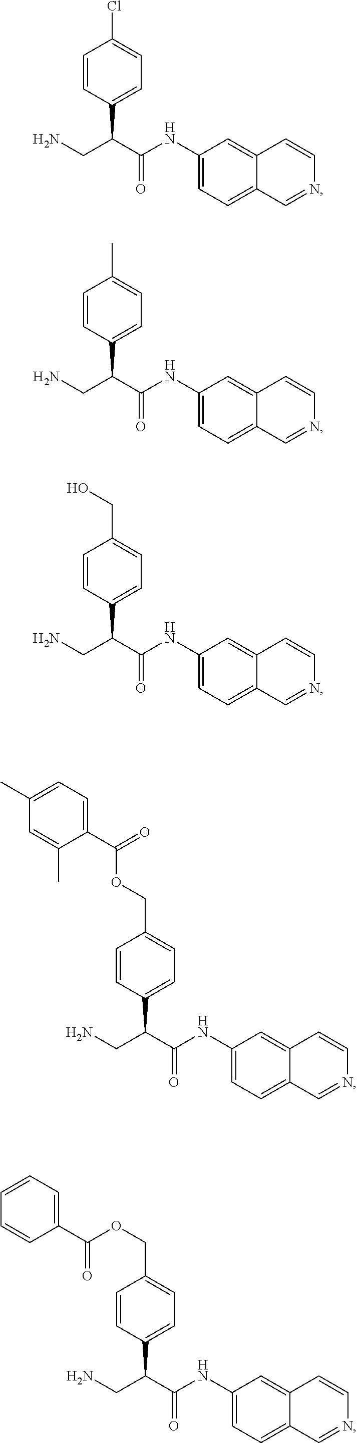 [SMILES: NC[C@H](C(=O)Nc1ccc2cnccc2c1)c1ccc(Cl)cc1 ; Cc1ccc([C@H](CN)C(=O)Nc2ccc3cnccc3c2)cc1 ; NC[C@H](C(=O)Nc1ccc2cnccc2c1)c1ccc(CO)cc1 ; Cc1ccc(C(=O)OCc2ccc([C@H](CN)C(=O)Nc3ccc4cnccc4c3)cc2)c(C)c1 ; NC[C@H](C(=O)Nc1ccc2cnccc2c1)c1ccc(COC(=O)c2ccccc2)cc1]

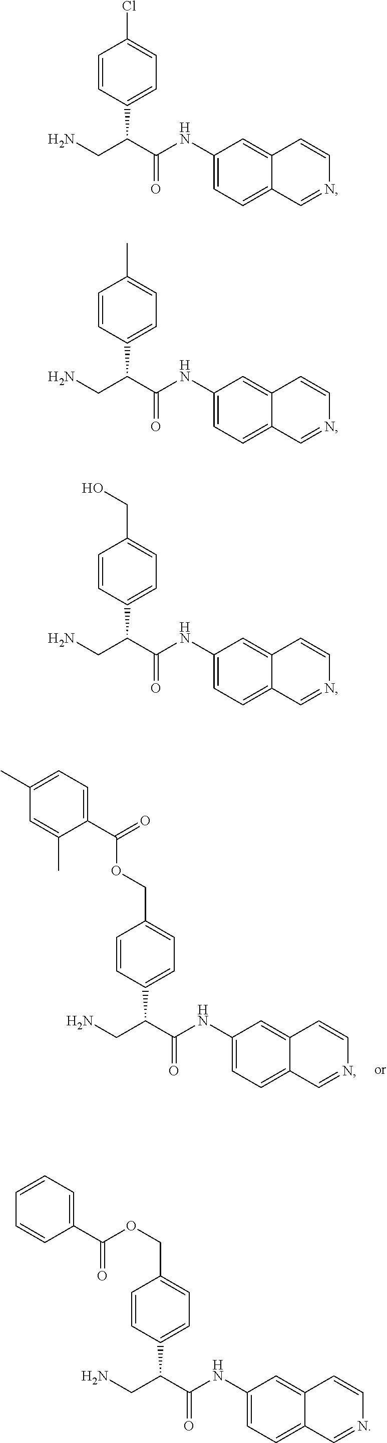 [SMILES: NC[C@@H](C(=O)Nc1ccc2cnccc2c1)c1ccc(Cl)cc1 ; Cc1ccc([C@@H](CN)C(=O)Nc2ccc3cnccc3c2)cc1 ; NC[C@@H](C(=O)Nc1ccc2cnccc2c1)c1ccc(CO)cc1 ; Cc1ccc(C(=O)OCc2ccc([C@@H](CN)C(=O)Nc3ccc4cnccc4c3)cc2)c(C)c1 ; "or" ; NC[C@@H](C(=O)Nc1ccc2cnccc2c1)c1ccc(COC(=O)c2ccccc2)cc1]

In some embodiments, the acid is p-toluenesulfonic acid, benzoic acid, benzenesulfonic acid, 2,4-dimethylbenzenesulfonic acid, phenylmethanesulfonic acid, 4-chlorobenzenesulfonic acid, [1,1′-biphenyl]-4-sulfonic acid, or cyclopentane sulfonic acid.

In some embodiments, the acid is p-toluenesulfonic acid, benzenesulfonic acid, 2,4-dimethylbenzenesulfonic acid, phenylmethanesulfonic acid, 4-chlorobenzenesulfonic acid, [1,1′-biphenyl]-4-sulfonic acid, or cyclopentane sulfonic acid.

The mono-(acid) salts provided herein may possess one or more stereocenters, and each stereocenter may exist independently in either the R or S configuration. In one embodiment, mono-(acid) salts described herein are present in optically active or racemic forms. It is to be understood that the mono-(acid) salts described herein encompass racemic, optically-active, regioisomeric and stereoisomeric forms, or combinations thereof that possess the therapeutically useful properties described herein.

Preparation of optically active forms is achieved in any suitable manner, including by way of non-limiting example, by resolution of the racemic form with recrystallization techniques, synthesis from optically-active starting materials, chiral synthesis, or chromatographic separation using a chiral stationary phase. In some embodiments, a mixture of one or more isomer is utilized as the therapeutic mono-(acid) salt described herein. In another embodiment, mono-(acid) salts described herein contain one or more chiral centers. These mono-(acid) salts are prepared by any means, including stereoselective synthesis, enantioselective synthesis and/or separation of a mixture of enantiomers or diastereomers. Resolution of mono-(acid) salts and isomers thereof is achieved by any means including, by way of non-limiting example, chemical processes, enzymatic processes, fractional crystallization, distillation, and chromatography.

Thus, in some embodiments, the compounds provided herein may be an enantiomer of the Formulae provided herein (e.g., the R enantiomer or the S enantiomer as shown below).

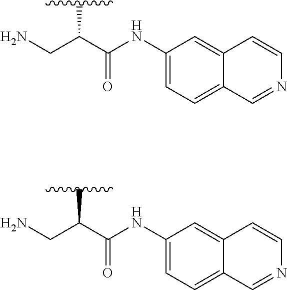 [SMILES: *[C@@H](CN)C(=O)Nc1ccc2cnccc2c1 ; *[C@H](CN)C(=O)Nc1ccc2cnccc2c1]

In some embodiments, the compounds may be in a composition (e.g., a pharmaceutical composition) as a combination of the enantiomers in any proportion to one another (e.g., about 0.1:99.9, 1:99, 1:50, 1:20, 1:10, 1:9, 1:8, 1:7, 1:6, 1:5, 1:4, 1:3, 1:2, 1:1, 2:1, 3:1, 4:1, 5:1, 6:1, 7:1, 8:1, 9:1, 10:1, 20:1, 50:1, 99:1, or 99.9:0.1 R:S as shown above).

In some embodiments, the mono-(acid) salts provided herein may exist as tautomers. All tautomers are included within the scope of the mono-(acid) salts presented herein.

Mono-(acid) salts described herein also include isotopically-labeled mono-(acid) salts wherein one or more atoms is replaced by an atom having the same atomic number, but an atomic mass or mass number different from the atomic mass or mass number usually found in nature. Examples of isotopes suitable for inclusion in the mono-(acid) salts described herein include and are not limited to ²H, ³H, ¹¹C, ¹³C, ¹⁴C, ³⁶Cl, ¹⁸F, ¹²³I, ¹²⁵I, ¹³N, ¹⁵N, ¹⁵O, ¹⁷O, ¹⁸O, ³²P and ³⁵S. In some embodiments, isotopically-labeled mono-(acid) salts are useful in drug or substrate tissue distribution studies. In some embodiments, substitution with heavier isotopes such as deuterium affords greater metabolic stability (for example, increased in vivo half-life or reduced dosage requirements). In yet another embodiment, substitution with positron emitting isotopes, such as ¹¹C, ¹⁸F, ¹⁵O and ¹³N, is useful in Positron Emission Topography (PET) studies for examining substrate receptor occupancy. Isotopically-labeled mono-(acid) salts are prepared by any suitable method or by processes using an appropriate isotopically-labeled reagent in place of the non-labeled reagent otherwise employed.

In some embodiments, the mono-(acid) salts described herein are labeled by other means, including, but not limited to, the use of chromophores or fluorescent moieties, bioluminescent labels, or chemiluminescent labels.

The mono-(acid) salts described herein, and other related mono-(acid) salts having different substituents are synthesized using techniques and materials described herein and as described, for example, in Fieser and Fieser's Reagents for Organic Synthesis, Volumes 1-17 (John Wiley and Sons, 1991); Rodd's Chemistry of Carbon Compounds, Volumes 1-5 and Supplementals (Elsevier Science Publishers, 1989); Organic Reactions, Volumes 1-40 (John Wiley and Sons, 1991), Larock's Comprehensive Organic Transformations (VCH Publishers Inc., 1989), March, Advanced Organic Chemistry 4th Ed., (Wiley 1992); Carey and Sundberg, Advanced Organic Chemistry 4th Ed., Vols. A and B (Plenum 2000, 2001), and Green and Wuts, Protective Groups in Organic Synthesis 3rd Ed., (Wiley 1999) (all of which are incorporated by reference for such disclosure). General methods for the preparation of mono-(acid) salts as described herein are modified by the use of appropriate reagents and conditions, for the introduction of the various moieties found in the formula as provided herein.

Mono-(acid) salts described herein are synthesized using any suitable procedures starting from compounds that are available from commercial sources, or are prepared using procedures described herein.

In some embodiment, reactive functional groups, such as hydroxyl, amino, imino, thio or carboxy groups, are protected in order to avoid their unwanted participation in reactions. Protecting groups are used to block some or all of the reactive moieties and prevent such groups from participating in chemical reactions until the protective group is removed. In another embodiment, each protective group is removable by a different means. Protective groups that are cleaved under totally disparate reaction conditions fulfill the requirement of differential removal.

### Solid Forms

In one aspect, provided herein is Form I of a mono-(hydrochloric acid) salt of (S)-4-(3-amino-1-(isoquinolin-6-ylamino)-1-oxopropan-2-yl)benzyl 2,4-dimethylbenzoate.

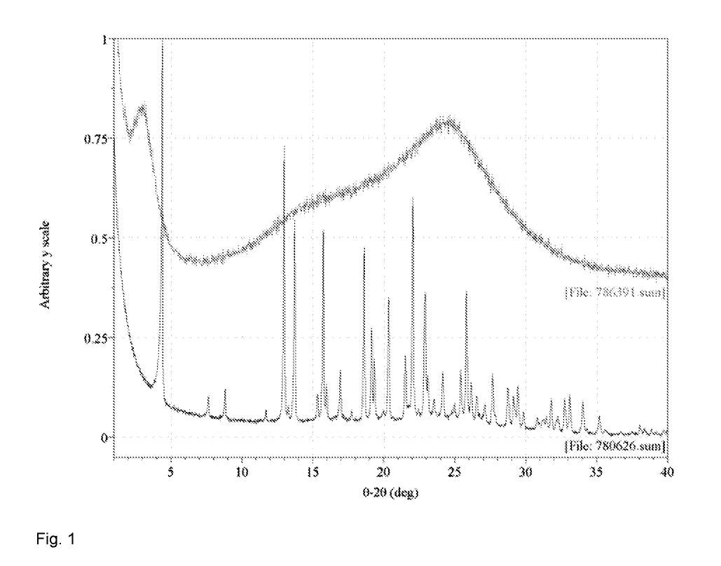

In some embodiments, the solid form has an X-ray powder diffraction pattern comprising a peak, in terms of 2-theta, at about 13.0°. In some embodiments, the solid form has an X-ray powder diffraction pattern comprising a peak, in terms of 2-theta, at about 13.0° and 22.1°. In some embodiments, the solid form has an X-ray powder diffraction pattern comprising a peak, in terms of 2-theta, at about 13.0°, 13.7°, 15.7°, 18.7°, and 22.1°. In some embodiments, the solid form has an X-ray powder diffraction pattern comprising a peak, in terms of 2-theta, at about 13.0°, 20.3°, 22.1°, 22.9°, and 25.8°. In some embodiments, the solid form has an X-ray powder diffraction pattern substantially as shown in FIG. 1.

In some embodiments, the solid form is substantially purified. In some embodiments, the solid form is crystalline.

The compounds provided herein also include polymorphs thereof. The terms “polymorph” or “polymorphism” as used herein refer to the ability of a solid material to exist in more than one form or crystal. A crystal form may be referred to herein as being characterized by graphical data. Such data include, for example, powder X-ray diffractograms and solid-state NMR spectra. As is well-known in the art, the graphical data potentially provides additional technical information to further define the respective solid state form (a so-called “fingerprint”) which can not necessarily be described by reference to numerical values or peak positions alone.

### Methods

Provided herein are methods of treating a disease in a subject in need thereof, comprising administering to the subject a therapeutically effective amount of the mono-(acid) salt of a compound of the Formulae provided herein.

Also provided herein are methods of treating a disease in a subject in need thereof, comprising administering to the subject a therapeutically effective amount of a composition provided herein.

Also provided herein are methods of treating a disease in a subject in need thereof, comprising administering to the subject a therapeutically effective amount of a pharmaceutical composition provided herein.

In some embodiments of these methods, the disease comprises glaucoma, an inflammatory eye disease, dry eye, ocular hypertension, or a neurodegenerative eye disease.

In some embodiments of these methods, the disease comprises diabetic macular edema, wet age-related macular degeneration, dry age-related macular degeneration, uveitis, glaucoma, and diabetic retinopathy.

In some embodiments, the disease comprises diabetic eye disease, wet age-related macular degeneration, or dry age-related macular degeneration.

In some embodiments, the disease is an ocular disorder.

In some embodiments, the ocular disorder is glaucoma, an inflammatory eye disease, dry eye, ocular hypertension, or a neurodegenerative eye disease.

In some embodiments, the ocular disorder is diabetic eye disease, wet age-related macular degeneration, or dry age-related macular degeneration.

Also provided herein are methods of reducing intraocular pressure in a subject in need thereof, comprising administering to the subject a therapeutically effective amount of the mono-(acid) salt of a compound of the Formulae provided herein.

Also provided herein are methods of reducing intraocular pressure in a subject in need thereof, comprising administering to the subject a therapeutically effective amount a composition provided herein.

Also provided herein are methods of reducing intraocular pressure in a subject in need thereof, comprising administering to the subject a therapeutically effective amount of a pharmaceutical composition provided herein.

In some embodiments of these methods, the administering to the subject is administering topically to an eye of the subject.

In some embodiments of these methods, the administering to the subject is administering topically to an eyelid of the subject.

Also provided herein are methods of inhibiting a kinase in a cell, comprising contacting the cell with an amount of the mono-(acid) salt of a compound of the Formulae provided herein effective to inhibit the kinase.

Also provided herein are methods of inhibiting a kinase in a cell, comprising contacting the cell with an amount of the mono-(acid) salt of a composition provided herein effective to inhibit the kinase.

Also provided herein are methods of inhibiting a kinase in a cell, comprising contacting the cell with an amount of the mono-(acid) salt of a pharmaceutical composition provided herein effective to inhibit the kinase.

Accordingly, in some embodiments, provided herein are methods of treating an ocular disorder in a subject in need thereof, comprising administering to the subject a therapeutically effective amount of the mono-(acid) salt of

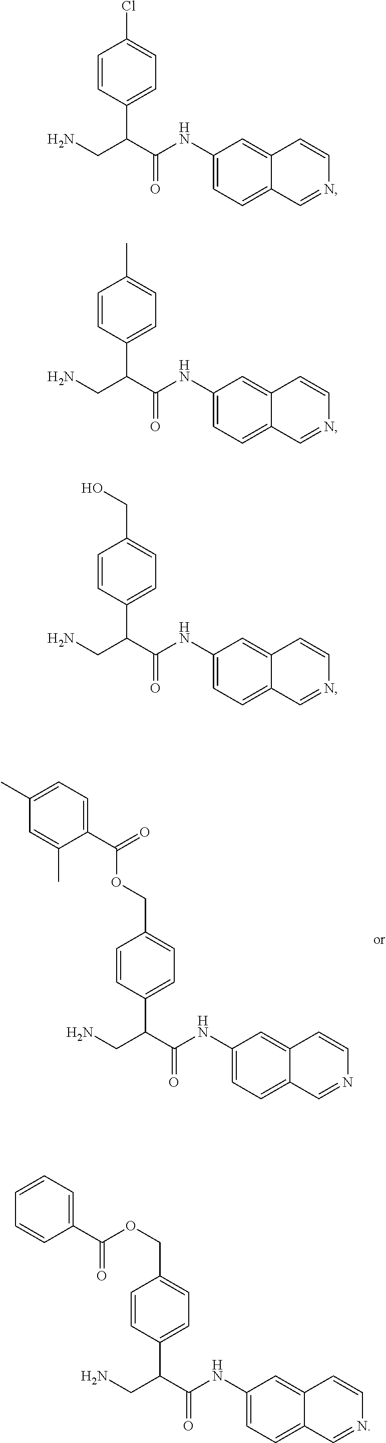 [SMILES: NCC(C(=O)Nc1ccc2cnccc2c1)c1ccc(Cl)cc1 ; Cc1ccc(C(CN)C(=O)Nc2ccc3cnccc3c2)cc1 ; NCC(C(=O)Nc1ccc2cnccc2c1)c1ccc(CO)cc1 ; Cc1ccc(C(=O)OCc2ccc(C(CN)C(=O)Nc3ccc4cnccc4c3)cc2)c(C)c1 ; NCC(C(=O)Nc1ccc2cnccc2c1)c1ccc(COC(=O)c2ccccc2)cc1]

In some embodiments, provided herein are methods of reducing intraocular pressure in a subject in need thereof, comprising administering to the subject a therapeutically effective amount of the mono-(acid) salt of

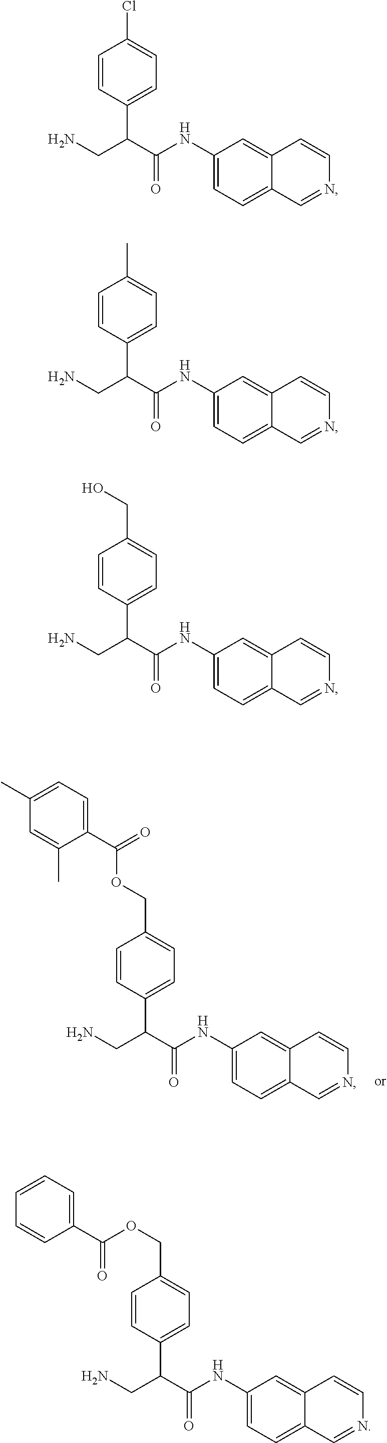 [SMILES: NCC(C(=O)Nc1ccc2cnccc2c1)c1ccc(Cl)cc1 ; Cc1ccc(C(CN)C(=O)Nc2ccc3cnccc3c2)cc1 ; NCC(C(=O)Nc1ccc2cnccc2c1)c1ccc(CO)cc1 ; Cc1ccc(C(=O)OCc2ccc(C(CN)C(=O)Nc3ccc4cnccc4c3)cc2)c(C)c1 ; "or" ; NCC(C(=O)Nc1ccc2cnccc2c1)c1ccc(COC(=O)c2ccccc2)cc1]

In some embodiments, provided herein are methods of modulating kinase activity in a cell, comprising contacting the cell with an amount of the mono-(acid) salt of

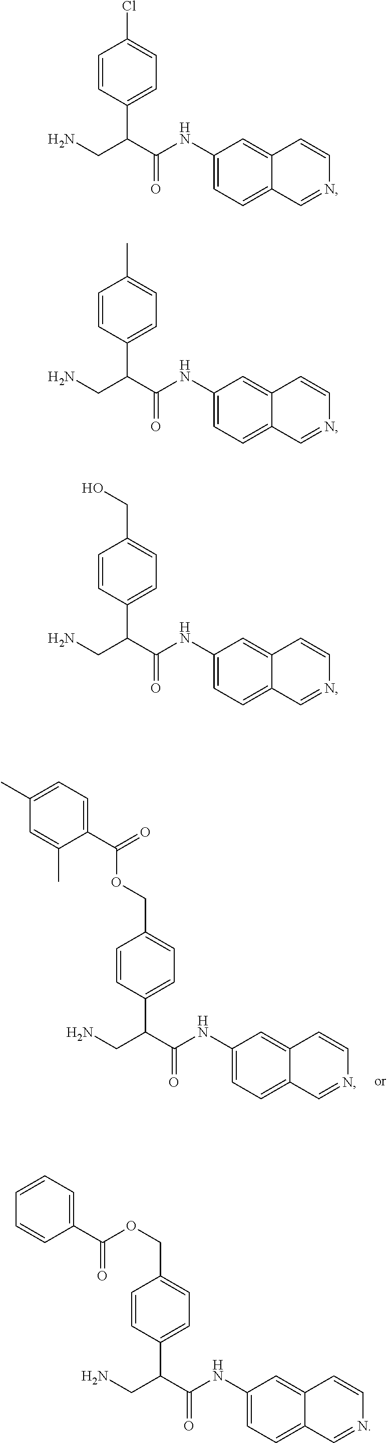 [SMILES: NCC(C(=O)Nc1ccc2cnccc2c1)c1ccc(Cl)cc1 ; Cc1ccc(C(CN)C(=O)Nc2ccc3cnccc3c2)cc1 ; NCC(C(=O)Nc1ccc2cnccc2c1)c1ccc(CO)cc1 ; Cc1ccc(C(=O)OCc2ccc(C(CN)C(=O)Nc3ccc4cnccc4c3)cc2)c(C)c1 ; "or" ; NCC(C(=O)Nc1ccc2cnccc2c1)c1ccc(COC(=O)c2ccccc2)cc1]

effective to inhibit the kinase.

In some embodiments, the cell is in a subject.

In some embodiments of these methods, the subject is a human subject.

### Compositions

Compositions may include one or more of the isoforms of the mono-(acid) salts of the Formulae provided herein when present. When racemates exists, each enantiomer or diastereomer may be separately used, or they may be combined in any proportion. Where tautomers exist all possible tautomers are specifically contemplated.

Pharmaceutical compositions for use in accordance with the present disclosure may be formulated in a conventional manner using one or more physiologically acceptable carriers or excipients. Thus, the mono-(acid) salts of the compounds of the Formulae provided herein may be formulated for administration by, for example, solid dosing, eyedrop, in a topical oil-based formulation, injection, inhalation (either through the mouth or the nose), implants, or oral, buccal, parenteral or rectal administration. Techniques and formulations may generally be found in “Remington's Pharmaceutical Sciences”, (Meade Publishing Co., Easton, Pa.). Therapeutic compositions must typically be sterile and stable under the conditions of manufacture and storage.

Thus, compositions are provided herein comprising a mono-(acid) salt of any of the Formulae provided herein.

In one aspect, provided herein are compositions, comprising a mono-(acid) salt of a compound of the Formulae provided herein, or a combination thereof.

In some embodiments, the composition comprises a mono-(acid) salt of a compound of the Formulae provided herein, or a combination thereof, and a polymeric matrix (e.g., a biodegradable polymeric matrix).

In some embodiments, the composition comprises about 1% to about 50% mono-(acid) salt by weight (e.g., about 1% to about 40%, about 1% to about 30%, about 1% to about 20%, about 1% to about 10%, about 10% to about 40%, about 10% to about 30%, or about 10% to about 20%).

In some embodiments, the composition comprises about 50% to about 99% polymeric matrix (e.g., about 60% to about 99%, about 70% to about 99%, about 80% to about 99%, about 90% to about 99%, about 60% to about 90%, about 70% to about 90%, or about 80% to about 90%).

In some embodiments, the composition comprises a ratio of about 1:99 to about 1:1 of mono-(acid) salt to polymeric matrix, respectively, by weight. In some embodiments, the composition comprises a ratio of about 1:9 to about 2:3 of mono-(acid) salt to polymeric matrix, respectively, by weight. In some embodiments, the composition comprises a ratio of about 1:99 to about 1:9 of mono-(acid) salt to polymeric matrix, respectively, by weight. In some embodiments, the composition comprises a ratio of about 1:9 to about 3:7 of mono-(acid) salt to polymeric matrix, respectively, by weight. In some embodiments, the composition comprises a ratio of about 1:5 to about 3:7 of mono-(acid) salt to polymeric matrix, respectively, by weight.

In some embodiments, the polymeric matrix is a biodegradable polymeric matrix.

In some embodiments, the composition comprises a combination of a mono-(acid) salt of Formula (Ia) and a mono-(acid) salt of Formula (Ib):

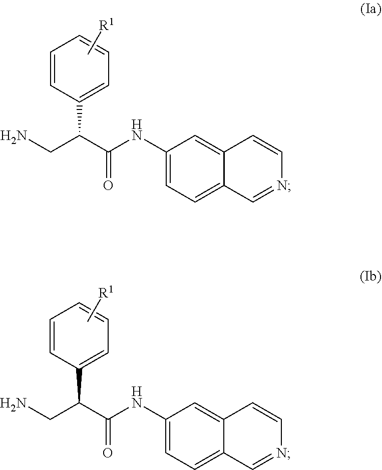

wherein
R¹ is H, halogen, —OH, —C₁₋₆ alkyl, —C₁₋₆ alkyl(R²)ₘ, —(CH₂)ₙOC(O)—(C₁₋₆ alkyl(R³)ₚ), or —(CH₂)ₙOC(O)—(C₆₋₁₀ aryl(R³)ₚ);
each R² is, independently, —OH or halogen;
each R³ is, independently, hydrogen, —OH, halogen, —C₁₋₆ alkyl, mono-halogen C₁₋₆ alkyl, di-halogen C₁₋₆ alkyl, or tri-halogen C₁₋₆ alkyl;
m is, independently, 1, 2, 3, 4, 5, or 6;
n is, independently, 1, 2, or 3; and
p is, independently, 0, 1, 2, 3, 4, 5, or 6.

In some embodiments, R¹ of Formula (Ia) and R¹ of Formula (Ib) are the same.

In some embodiments, the composition comprises a combination of a mono-(acid) salt of (R)-3-amino-2-(4-(hydroxymethyl)phenyl)-N-(isoquinolin-6-yl)propanamide and a mono-(acid) salt of (S)-3-amino-2-(4-(hydroxymethyl)phenyl)-N-(isoquinolin-6-yl)propanamide.

In some embodiments, the composition comprises a mono-(acid) salt of (rac)-3-amino-2-(4-(hydroxymethyl)phenyl)-N-(isoquinolin-6-yl)propanamide, (R)-3-amino-2-(4-(hydroxymethyl)phenyl)-N-(isoquinolin-6-yl)propanamide, or a mono-(acid) salt of (S)-3-amino-2-(4-(hydroxymethyl)phenyl)-N-(isoquinolin-6-yl)propanamide.

In some embodiments, the composition comprises a combination of a mono-(acid) salt of (R)-3-amino-2-(4-chlorophenyl)-N-(isoquinolin-6-yl)propanamide and a mono-(acid) salt of (S)-3-amino-2-(4-chlorophenyl)-N-(isoquinolin-6-yl)propanamide.

In some embodiments, the composition comprises a mono-(acid) salt of (rac)-3-amino-2-(4-chlorophenyl)-N-(isoquinolin-6-yl)propanamide, (R)-3-amino-2-(4-chlorophenyl)-N-(isoquinolin-6-yl)propanamide, or a mono-(acid) salt of (S)-3-amino-2-(4-chlorophenyl)-N-(isoquinolin-6-yl)propanamide.

In some embodiments, the composition comprises a combination of a mono-(acid) salt of (R)-3-amino-N-(isoquinolin-6-yl)-2-(p-tolyl)propanamide and a mono-(acid) salt of (S)-3-amino-N-(isoquinolin-6-yl)-2-(p-tolyl)propanamide.

In some embodiments, the composition comprises a mono-(acid) salt of (rac)-3-amino-N-(isoquinolin-6-yl)-2-(p-tolyl)propanamide, (R)-3-amino-N-(isoquinolin-6-yl)-2-(p-tolyl)propanamide, or a mono-(acid) salt of (S)-3-amino-N-(isoquinolin-6-yl)-2-(p-tolyl)propanamide.

In some embodiments, the composition comprises a combination of a mono-(acid) salt of (R)-4-(3-amino-1-(isoquinolin-6-ylamino)-1-oxopropan-2-yl)benzyl 2,4-dimethylbenzoate and a mono-(acid) salt of (S)-4-(3-amino-1-(isoquinolin-6-ylamino)-1-oxopropan-2-yl)benzyl 2,4-dimethylbenzoate.

In some embodiments, the composition comprises a mono-(acid) salt of (rac)-4-(3-amino-1-(isoquinolin-6-ylamino)-1-oxopropan-2-yl)benzyl 2,4-dimethylbenzoate, (R)-4-(3-amino-1-(isoquinolin-6-ylamino)-1-oxopropan-2-yl)benzyl 2,4-dimethylbenzoate, or a mono-(acid) salt of (S)-4-(3-amino-1-(isoquinolin-6-ylamino)-1-oxopropan-2-yl)benzyl 2,4-dimethylbenzoate.

In some embodiments, the composition comprises a combination of a mono-(acid) salt of (R)-4-(3-amino-1-(isoquinolin-6-ylamino)-1-oxopropan-2-yl)benzyl benzoate and a mon-(acid) salt of (S)-4-(3-amino-1-(isoquinolin-6-ylamino)-1-oxopropan-2-yl)benzyl benzoate

In some embodiments, the composition comprises a mono-(acid) salt of (rac)-4-(3-amino-1-(isoquinolin-6-ylamino)-1-oxopropan-2-yl)benzyl benzoate, (R)-4-(3-amino-1-(isoquinolin-6-ylamino)-1-oxopropan-2-yl)benzyl benzoate, or a mono-(acid) salt of (S)-4-(3-amino-1-(isoquinolin-6-ylamino)-1-oxopropan-2-yl)benzyl benzoate

In some embodiments, the compositions are pharmaceutical compositions further comprising a pharmaceutically acceptable carrier.

Compositions provided herein may comprise a safe and effective amount of the subject mono-(acid) salts, and a pharmaceutically-acceptable carrier.

The route by which the mono-(acid) salts, compositions, or pharmaceutical compositions provided herein (component A) will be administered and their form will dictate the type of carrier (component B) to be used. Component A may be in a variety of forms, suitable, for example, for systemic administration (e.g., oral, rectal, nasal, sublingual, buccal, implants, or parenteral) or topical administration (e.g., local application on the skin, ocular, liposome delivery systems, or iontophoresis).

Carriers for systemic administration typically comprise at least one of a) diluents, b) lubricants, c) binders, d) disintegrants, e) colorants, f) flavors, g) sweeteners, h) antioxidants, j) preservatives, k) glidants, m) solvents, n) suspending agents, o) wetting agents, p) surfactants, q) biodegradable polymers, r) plasticizers, combinations thereof, and others. All carriers are optional in the systemic compositions.

Although the amounts of components A and B in the systemic compositions will vary depending on the type of systemic composition prepared, the specific derivative selected for component A and the ingredients of component B, in general, system compositions comprise 0.01% to 50% of component A and 50 to 99.99% of component B.

Compositions for parenteral administration typically comprise A) 0.1 to 10% of the mono-(acid) salts provided herein and B) 90 to 99.9% of a carrier comprising a) a diluent and m) a solvent. In one embodiment, component a) comprises propylene glycol and m) comprises ethanol or ethyl oleate.

Compositions for oral administration can have various dosage forms. For example, solid forms include tablets, capsules, granules, and bulk powders. These oral dosage forms comprise a safe and effective amount, usually at least about 5%, and more particularly from about 25% to about 50% of component A). The oral dosage compositions further comprise about 50 to about 95% of component B), and more particularly, from about 50 to about 75%.

Tablets can be compressed, tablet triturates, enteric-coated, sugar-coated, film-coated, or multiple-compressed. Tablets typically comprise component A, and component B a carrier comprising ingredients selected from the group consisting of a) diluents, b) lubricants, c) binders, d) disintegrants, e) colorants, f) flavors, g) sweeteners, k) glidants, and combinations thereof.

Capsules (including implants, time release and sustained release formulations) typically comprise component A, and a carrier comprising one or more a) diluents disclosed above in a capsule comprising gelatin. Granules typically comprise component A, and preferably further comprise k) glidants such as silicon dioxide to improve flow characteristics. Implants can be of the biodegradable or the non-biodegradable type. Implants may be prepared using any known biocompatible formulation.

The selection of ingredients in the carrier for oral compositions depends on secondary considerations like taste, cost, and shelf stability, which are not critical for the purposes of this disclosure. One skilled in the art would know how to select appropriate ingredients without undue experimentation.

The solid compositions may also be coated by conventional methods, typically with pH or time-dependent coatings, such that component A is released in the gastrointestinal tract in the vicinity of the desired application, or at various points and times to extend the desired action. The coatings typically comprise one or more components selected from the group consisting of cellulose acetate phthalate, polyvinyl acetate phthalate, hydroxypropyl methyl cellulose phthalate, ethyl cellulose, EUDRAGIT® coatings (available from Rohm & Haas G.M.B.H. of Darmstadt, Germany), waxes and shellac.

Compositions for oral administration can also have liquid forms. For example, suitable liquid forms include aqueous solutions, emulsions, suspensions, solutions reconstituted from non-effervescent granules, suspensions reconstituted from non-effervescent granules, effervescent preparations reconstituted from effervescent granules, elixirs, tinctures, syrups, and the like. Liquid orally administered compositions typically comprise component A and component B, namely, a carrier comprising ingredients selected from the group consisting of a) diluents, e) colorants, f) flavors, g) sweeteners, j) preservatives, m) solvents, n) suspending agents, and o) surfactants. Peroral liquid compositions preferably comprise one or more ingredients selected from the group consisting of e) colorants, f) flavors, and g) sweeteners.

Other compositions useful for attaining systemic delivery of the subject mono-(acid) salts include implanted, sublingual, buccal and nasal dosage forms. Such compositions typically comprise one or more of soluble or biodegradable filler substances such as a) diluents including sucrose, sorbitol and mannitol; and c) binders such as acacia, microcrystalline cellulose, carboxymethyl cellulose, and hydroxypropyl methylcellulose. Such compositions may further comprise b) lubricants, e) colorants, f) flavors, g) sweeteners, h) antioxidants, and k) glidants. Implanted formulations typically include q) biodegradable polymers and optionally, r) plasticizers.

In one embodiment of the disclosure, the mono-(acid) salts provided herein are topically administered. Topical compositions that can be applied locally to the eye may be in any form known in the art, non-limiting examples of which include solids, gelable drops, sprays, ointments, or a sustained or non-sustained release unit placed in the conjunctival cul-du-sac of the eye, in the intracameral space, in the aqueous humor, in the vitreous humor, or another appropriate location.

Topical compositions that can be applied locally to the skin may be in any form including solids, solutions, oils, creams, ointments, gels, lotions, shampoos, leave-on and rinse-out hair conditioners, milks, cleansers, moisturizers, sprays, skin patches, and the like. Topical compositions comprise: component A, the mono-(acid) salts described above, and component B, a carrier. The carrier of the topical composition preferably aids penetration of the mono-(acid) salts into the eye. Component B may further comprise one or more optional components.

The dosage range of the mono-(acid) salt for systemic administration is from about 0.01 to about 1000 μg/kg body weight, preferably from about 0.1 to about 100 μg/kg per body weight, most preferably from about 1 to about 50 μg/kg body weight per day. While these dosages are based upon a daily administration rate, weekly or monthly accumulated dosages may also be used to calculate the clinical requirements.

The dosage range for direct injection in a slowly-releasing dose form into the back of the eye is from about 0.1 ng to about 5 ug per eye per day, preferably from 1000 ng to 1 ng per eye per day, more preferably from about 500 to 50 ng/eye/day. Note that a loading dose above this range might be desirable in the case where the injection is designed to last for months. This loading dose would be in the range of 1000 ng to 10 ng per eye per day for the first 1-10 days of dosing, with the dose level falling to the above ranges afterward.

Dosages may be varied based on the patient being treated, the disease or disorder being treated, the severity of the disease or disorder being treated, the route of administration, etc. to achieve the desired effect.

The mono-(acid) salts provided herein are useful in decreasing intraocular pressure. Thus, these mono-(acid) salts are useful in the treatment of glaucoma. One route of administration for treating glaucoma is topically.

The exact amounts of each component in the topical composition depend on various factors. The amount of component A added to the topical composition is dependent on the IC₅₀ of component A, typically expressed in nanomolar (nM) units. For example, if the IC₅₀ of the medicament is 1 nM, the amount of component A will be from about 0.001 to about 0.3%. If the IC₅₀ of the medicament is 10 nM, the amount of component A) will be from about 0.01 to about 1%. If the IC₅₀ of the medicament is 100 nM, the amount of component A will be from about 0.1 to about 10%. If the IC₅₀ of the medicament is 1000 nM, the amount of component A will be 1 to 100%, preferably 5% to 50%. If the amount of component A is outside the ranges specified above (i.e., lower), efficacy of the treatment may be reduced. One skilled in the art understands how to calculate and understand an IC₅₀. The remainder of the composition, up to 100%, is component B.

The amount of the carrier employed in conjunction with component A is sufficient to provide a practical quantity of composition for administration per unit dose of the medicament. Techniques and compositions for making dosage forms useful in the methods of this disclosure are described in the following references: Modern Pharmaceutics, Chapters 9 and 10, Banker & Rhodes, eds. (1979); Lieberman et al., Pharmaceutical Dosage Forms: Tablets (1981); and Ansel, Introduction to Pharmaceutical Dosage Forms, 2^{nd} Ed., (1976).

Component B may comprise a single ingredient or a combination of two or more ingredients. In the topical compositions, component B comprises a topical carrier.

The carrier of the topical composition may further comprise one or more ingredients selected from the group consisting of q) emollients, r) propellants, s) solvents, t) humectants, u) thickeners, v) powders, w) fragrances, x) pigments, and y) preservatives.

Component A may be included in kits comprising component A, a systemic or topical composition described above, or both; and information, instructions, or both that use of the kit will provide treatment for cosmetic and medical diseases or disorders in mammals (particularly humans). The information and instructions may be in the form of words, pictures, or both, and the like. In addition or in the alternative, the kit may comprise the medicament, a composition, or both; and information, instructions, or both, regarding methods of application of medicament, or of composition, preferably with the benefit of treating or preventing cosmetic and medical diseases or disorders in mammals (e.g., humans). Component A may also be included in articles of manufacture for use as described herein for mono-(acid) salts provided herein.

Thus, provided herein are kits comprising a mono-(acid) salt of a compound of any of the Formulae provided herein, and instructions for use thereof. Also provided herein are kits comprising a composition provided herein, and instructions for use thereof. Also provided herein are kits comprising a pharmaceutical composition provided herein, and instructions for use thereof.

Also provided herein are articles of manufacture comprising a mono-(acid) salt of a compound of any of the Formulae provided herein. Also provided herein are articles of manufacture comprising a composition provided herein, and instructions for use thereof. Also provided herein are articles of manufacture comprising a pharmaceutical composition provided herein, and instructions for use thereof.

### EXAMPLES

The following illustrative examples are to be considered non-limiting. Procedures for the preparation of mono-(acid) salts of the Formulae provided herein are described in the following examples.

All temperatures are in degrees Centigrade. Reagents and starting materials were purchased from commercial sources or prepared following published literature procedures.

Unless otherwise noted, HPLC purification, when appropriate, was performed by redissolving the compound in a small volume of DMSO and filtering through a 0.45 micron (nylon disc) syringe filter. The solution was then purified using, for example, a 50 mm Varian Dynamax HPLC 21.4 mm Microsorb Guard-8 C₈ column. A typical initial eluting mixture of 40-80% MeOH:H₂O was selected as appropriate for the target compound. This initial gradient was maintained for 0.5 minutes then increased to 100% MeOH: 0% H₂O over 5 minutes. 100% MeOH was maintained for 2 more minutes before re-equilibration back to the initial starting gradient. A typical total run time was 8 minutes. The resulting fractions were analyzed, combined as appropriate, and then evaporated to provide purified material.

Proton magnetic resonance (¹H NMR) spectra were recorded on either a Varian INOVA 600 MHz (¹H) NMR spectrometer, Varian INOVA 500 MHz (¹H) NMR spectrometer, Varian Mercury 300 MHz (¹H) NMR spectrometer, or a Varian Mercury 200 MHz (¹H) NMR spectrometer. All spectra have been determined in the solvents indicated. Although chemical shifts are reported in ppm downfield of tetramethylsilane, they are referenced to the residual proton peak of the respective solvent peak for ¹H NMR. Interproton coupling constants are reported in Hertz (Hz).

Analytical LCMS spectra were obtained using a Waters Acquity QDA MS ESI instrument with an Alliance 2695 HPLC and a998 Photodiode Array Detector. Spectra were analyzed at 254 and 230 nm. Samples were passed through a Waters Atlantis T3 4.6×75 mm Column with a guard column (Atlantis T3 4.6×20 mm) Gradients were run with mobile phase A: 0.1% formic acid in H₂O and mobile phase B: 0.1% formic aci in ACN with a flow rate of 0.8 mL/min. Gradient A (Table 1) is illustrative of a gradient used for analytical LCMS.

TABLE 1
Gradient A

| | |
|---|---|
| Gradient A | Gradient B |

| Time (minutes) | A % | B % | A % | B % |
|---|---|---|---|---|
| 0.00 | 95.0 | 5.0 | 80.0 | 20.0 |
| 1.00 | 95.0 | 5.0 | 80.0 | 20.0 |
| 6.00 | 40.0 | 60.0 | 25.0 | 75.0 |
| 7.00 | 5.0 | 95.0 | 5.0 | 95.0 |
| 8.00 | 5.0 | 95.0 | 5.0 | 95.0 |
| 9.00 | 95.0 | 5.0 | 80.0 | 20.0 |
| 12.00 | 95.0 | 5.0 | 80.0 | 20.0 |

The settings for the MS probe were a cone voltage at 15 V, capillary voltage at 0.8 KV for Positive mode and 0.4 kV for negative mode. The probe temperature is 600° C. and the source temperature is 120° C. Any variations in these methods are noted below.

The following examples illustrate procedures for the preparation of intermediates and methods for the preparation of mono-(acid) salts provided herein.

### Example 1. Synthesis of the mono-(hydrochloric acid) salt of (S)-4-(3-amino-1-(isoquinolin-6-ylamino)-1-oxopropan-2-yl)benzyl 2,4-dimethylbenzoate (Scheme 1)

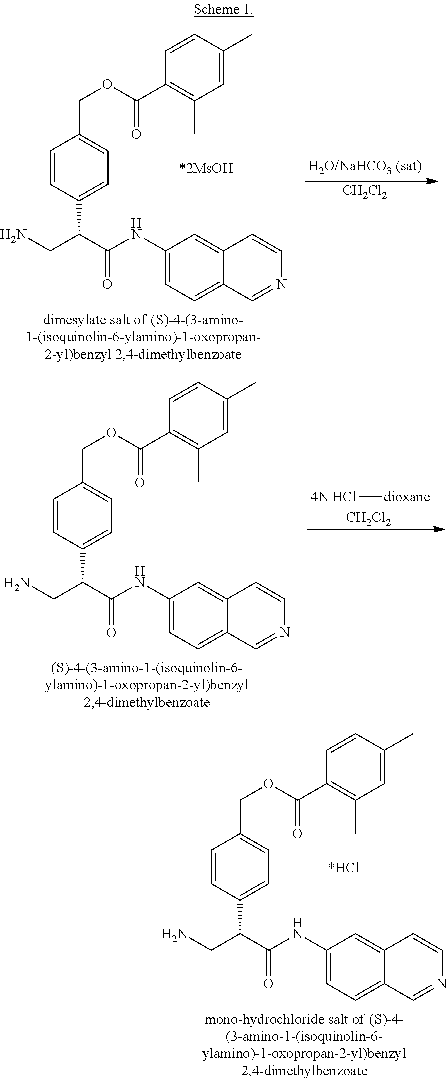

### Preparation of (S)-4-(3-amino-1-(isoquinolin-6-ylamino)-1-oxopropan-2-yl)benzyl 2,4-dimethylbenzoate

The dimesylate salt of (S)-4-(3-amino-1-(isoquinolin-6-ylamino)-1-oxopropan-2-yl)benzyl 2,4-dimethylbenzoate (20.5 g, 31.75 mmol) was dissolved in water (80 mL) and treated with a NaHCO₃ saturated solution (55 mL) portion wise over 10 minutes until pH 7.5. The resulting slurry was stirred 20 minutes and then extracted with CH₂Cl₂/EtOH (500 mL/30 mL) and CH₂Cl₂ (4×100 mL). The organic layers were washed with brine (50 mL), dried over Na₂SO₄ and concentrated to give 15.3 g (S)-4-(3-amino-1-(isoquinolin-6-ylamino)-1-oxopropan-2-yl)benzyl 2,4-dimethylbenzoate. The product was dried on a high vacuum overnight at 30° C. ¹H-NMR showed that some solvent was still present in the product which was split on 2 portions: 9 g was moved to the next step and the rest (5.1 g) was dried during 3 days under high vacuum at 40° C. The latter one provided the first target compound, (S)-4-(3-amino-1-(isoquinolin-6-ylamino)-1-oxopropan-2-yl)benzyl 2,4-dimethylbenzoate (5 g). The total yield for this step was 95%.

### Preparation of mono-(hydrochloric acid) salt of (S)-4-(3-amino-1-(isoquinolin-6-ylamino)-1-oxopropan-2-yl)benzyl 2,4-dimethylbenzoate

A solution of (S)-4-(3-amino-1-(isoquinolin-6-ylamino)-1-oxopropan-2-yl)benzyl 2,4-dimethylbenzoate (9.0 g, 19.84 mmol) in dry CH₂Cl₂ (100 mL) was treated with 4N HCl in dioxane (1 eq, 4.96 mL, 19.84 mmol) over 5 minutes under N₂ and stirring. The resulting slurry was left to stir overnight. The reaction mixture was concentrated with toluene (2×50 mL) and dried on a high vacuum for 3 days at 40° C. to give mono-(hydrochloric acid) salt of (S)-4-(3-amino-1-(isoquinolin-6-ylamino)-1-oxopropan-2-yl)benzyl 2,4-dimethylbenzoate (9.6 g, 97%).

### Example 2. Synthesis of mono-(hydrochloric acid) salt of (S)-4-(3-amino-1-(isoquinolin-6-ylamino)-1-oxopropan-2-yl)benzyl 2,4-dimethylbenzoate (Scheme 2)

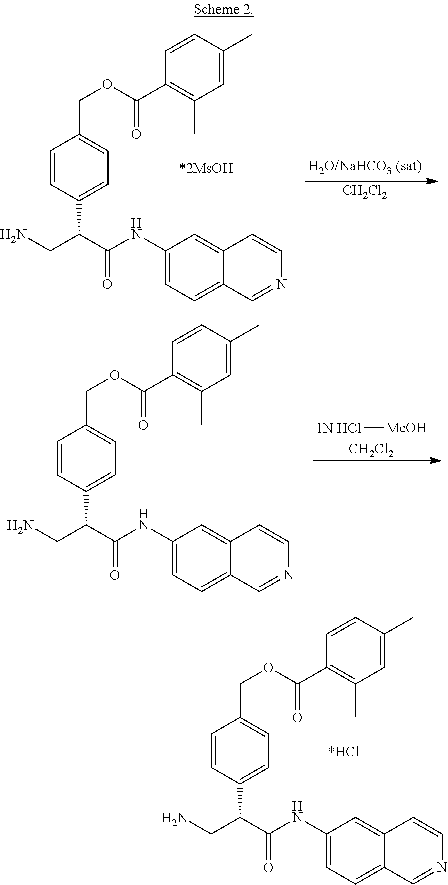

### Preparation of (S)-4-(3-amino-1-(isoquinolin-6-ylamino)-1-oxopropan-2-yl)benzyl 2,4-dimethylbenzoate

To the dimesylate salt of (S)-4-(3-amino-1-(isoquinolin-6-ylamino)-1-oxopropan-2-yl)benzyl 2,4-dimethylbenzoate (2.5 g, 3.8 mmol) was added a minimum amount of water to dissolve. Then NaHCO₃(sat) was added to precipitate out. The aqueous layer was extracted several times with CH₂Cl₂ and a little MeOH to recover (S)-4-(3-amino-1-(isoquinolin-6-ylamino)-1-oxopropan-2-yl)benzyl 2,4-dimethylbenzoate (2.7 g, 94%).

### Preparation of 1N HCl-MeOH

To MeOH (9.3 mL) cooled to 0° C. was added acetyl chloride (710 μL, 10 mmol) and the solution was stirred for 10 minutes at 0° C.

### Preparation of mono-(hydrochloric acid) salt of (S)-4-(3-amino-1-(isoquinolin-6-ylamino)-1-oxopropan-2-yl)benzyl 2,4-dimethylbenzoate

(S)-4-(3-amino-1-(isoquinolin-6-ylamino)-1-oxopropan-2-yl)benzyl 2,4-dimethylbenzoate (1.0 g, 2.2 mmol) was dissolved in CH₂Cl₂ (30 mL) and HCl-MeOH (1N, 2.2 mL, 2.2 mmol) was added to the reaction (dropwise quickly). A precipitate formed and the mixture was stirred an additional 20 min. The solvents were evaporated and the compound was dried on the high vacuum for approx. 60 hours to give mono-(hydrochloric acid) salt of (S)-4-(3-amino-1-(isoquinolin-6-ylamino)-1-oxopropan-2-yl)benzyl 2,4-dimethylbenzoate (1.04 g, 97%).

### Example 3. Synthesis of di-(hydrochloric acid) salt of (S)-4-(3-amino-1-(isoquinolin-6-ylamino)-1-oxopropan-2-yl)benzyl 2,4-dimethylbenzoate (Scheme 3)

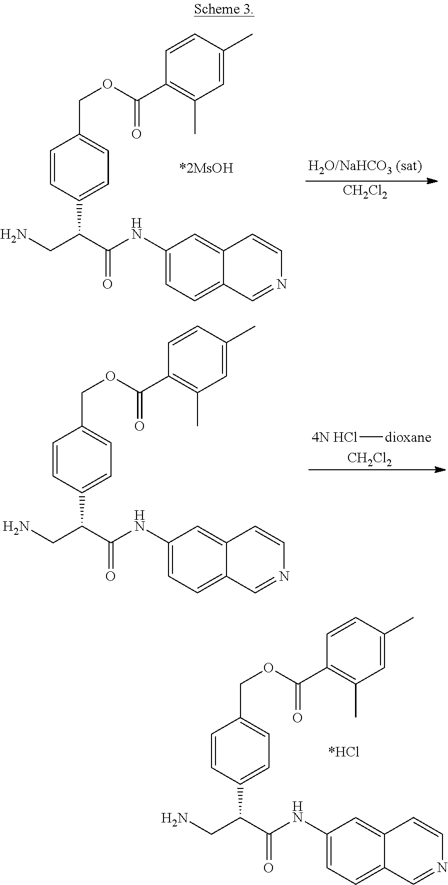

### Preparation of di-(hydrochloric acid) salt of (S)-4-(3-amino-1-(isoquinolin-6-ylamino)-1-oxopropan-2-yl)benzyl 2,4-dimethylbenzoate

A solution of (S)-4-(3-amino-1-(isoquinolin-6-ylamino)-1-oxopropan-2-yl)benzyl 2,4-dimethylbenzoate (250 mg, 0.55 mmol) in dry CH₂Cl₂ (3.2 mL) was treated with 4N HCl in dioxane (2.5 eq, 343 μL, 1.38 mmol). After 2 hours the solvents were evaporated and the solids were azeotroped with toluene (3×3 mL) and dried on the high vacuum for 4 days at 40° C. to give di-(hydrochloric acid) salt of (S)-4-(3-amino-1-(isoquinolin-6-ylamino)-1-oxopropan-2-yl)benzyl 2,4-dimethylbenzoate (268 mg, 93%).

### Example 4. Melting Point and X-Ray Powder Diffraction (XRPD)

Melting points and XRPD patterns were collected for compounds as described in Table 2. As can be seen from Table 2, it was unexpectedly found that the mono-(acid) salt (e.g., mono-(hydrochloric acid) salt of (S)-4-(3-amino-1-(isoquinolin-6-ylamino)-1-oxopropan-2-yl)benzyl 2,4-dimethylbenzoate) had significantly higher melting point (≥40° C.) than the corresponding di-salt, and the mono-(acid) salt had a significantly higher melting point (>150° C.) than the corresponding free base. Additionally, the mono-(acid) salt was surprisingly found to be crystalline whereas the corresponding di-salt was amorphous (see FIG. 1). Without being bound by theory, these unexpected and surprising properties would render the mono-(acid) salt form more suitable to manufacturing processes relative to the corresponding free-base form or the corresponding di-salt form because the mono-(acid) salt form would be able to withstand more rigorous manufacturing methods (e.g., increased temperature, pressure, etc.) due to the enhanced thermal stability of a mono-(acid) salt.

TABLE 2

| 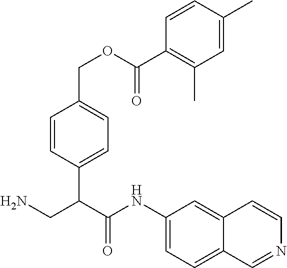 [SMILES: Cc1ccc(C(=O)OCc2ccc(C(CN)C(=O)Nc3ccc4cnccc4c3)cc2)c(C)c1] | Melting point | XRPD Analysis Results |
|---|---|---|
| free base | 58-68° C. | Not Determined |
| HCl salt | 221-226° C. | Crystalline |
| di-HCl salt | 171-181° C. | Amorphous |
| | (liquid at 190° C.) | |

### Example 5. Synthesis of mono-(acid) salts of racemic 4-(3-amino-1-(isoquinolin-6-ylamino)-1-oxopropan-2-yl)benzyl 2,4-dimethylbenzoate

### General Procedure:

To 4-(3-amino-1-(isoquinolin-6-ylamino)-1-oxopropan-2-yl)benzyl 2,4-dimethylbenzoate in MeOH-EtOAc was added acid (neat or dissolved in EtOAc) and the reaction was stirred at room temperature for 0.5-12 hours. The solvents were evaporated and dried to give the corresponding mono-(acid) salts of 4-(3-amino-1-(isoquinolin-6-ylamino)-1-oxopropan-2-yl)benzyl 2,4-dimethylbenzoate. Non-limiting examples of mono-(acid) salts prepared by this procedure are shown in Table 3 and Table 4.

TABLE 3

| | MW or | | | | |
|---|---|---|---|---|---|
| | molarity | Amount | mmol | equivalents | Yield |
| 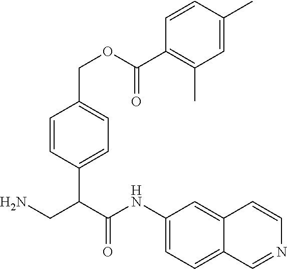 [SMILES: Cc1ccc(C(=O)OCc2ccc(C(CN)C(=O)Nc3ccc4cnccc4c3)cc2)c(C)c1] | 453.53 | 40 mg | 0.088 mmol | | |
| methanesulfonic acid | 96.11 | 5.3 μL | 0.088 mmol | 1.0 | 32 mg, 67% |
| p-toluenesulfonic acid monohydrate | 190.20 | 16.8 mg | 0.088 mmol | 1.0 | 49 mg, 89%, |
| benzoic acid | 122.12 | 10.8 mg | 0.088 mmol | 1.0 | 33 mg, 65% |
| sulfuric acid | 36.0 M | 2.4 μL | 0.088 mmol | 1.0 | 40 mg, 82% |
| HCl | 12 M | 7.3 μL | 0.088 mmol | 1.0 | 35 mg, 82% |
| acetic acid | 17.4 M | 5.1 μL | 0.088 mmol | 1.0 | 40 mg, 89% |
| phosphoric acid | 14.7 M | 6.0 μL | 0.088 mmol | 1.0 | 24 mg, 50% |
| citric acid | 192.12 | 16.9 mg | 0.088 mmol | 1.0 | 45 mg, 80% |
| benzenesulfonic acid monohydrate | 176.18 | 15.5 mg | 0.088 mmol | 1.0 | 28 mg, 53% |
| sorbate | 112.13 | 10 mg | 0.088 mmol | 1.0 | 43 mg, 86% |
| L-aspartate | 133.10 | 12 mg | 0.088 mmol | 1.0 | 53 mg, >99% |

TABLE 4

| | MW or | | | | |
|---|---|---|---|---|---|
| | molarity | Amount | mmol | equivalents | Yield |
| 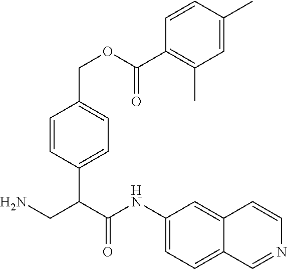 [SMILES: Cc1ccc(C(=O)OCc2ccc(C(CN)C(=O)Nc3ccc4cnccc4c3)cc2)c(C)c1] | 453.53 | 40 mg | 0.088 mmol | | |
| tartaric acid | 150.09 | 25 mg | 0.165 | 1.0 | 89 mg, 90% |
| fumaric acid | 116.07 | 19 mg | 0.165 | 1.0 | 100 mg, >99% |
| maleic acid | 116.07 | 19 mg | 0.165 | 1.0 | 80 mg, 85% |

### Example 6. Synthesis of mono-(acid) salts of racemic 4-(3-amino-1-(isoquinolin-6-ylamino)-1-oxopropan-2-yl)benzyl 2,4-dimethylbenzoate

### General Procedure:

To a solution of 4-(3-amino-1-(isoquinolin-6-ylamino)-1-oxopropan-2-yl)benzyl 2,4-dimethylbenzoate in MeOH was added a suspension of L-aspartic acid in water and solution was stirred overnight at room temperature. The solvents were evaporated and the solids were azeotroped with benzene and dried on the high vacuum to give the mono-(L-aspartic acid) salt of 4-(3-amino-1-(isoquinolin-6-ylamino)-1-oxopropan-2-yl)benzyl 2,4-dimethylbenzoate. A non-limiting example of a mono-(acid) salt prepared by this procedure is shown in Table 5.

TABLE 5

| | MW | Amount | mmol | equivalents | Yield |
|---|---|---|---|---|---|
| 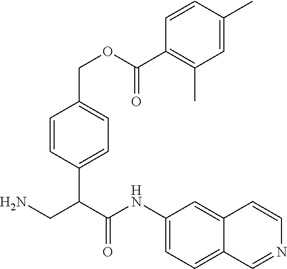 [SMILES: Cc1ccc(C(=O)OCc2ccc(C(CN)C(=O)Nc3ccc4cnccc4c3)cc2)c(C)c1] | 453.53 | 71 mg | 0.16 mmol | | |
| L-aspartic acid | 133.10 | 21 mg | 0.16 mmol | 1.0 | 89 mg, 96% |

### Example 7. Synthesis of mono-(acid) salts of racemic 4-(3-amino-1-(isoquinolin-6-ylamino)-1-oxopropan-2-yl)benzyl 2,4-dimethylbenzoate

### General Procedure:

To a solution of 4-(3-amino-1-(isoquinolin-6-ylamino)-1-oxopropan-2-yl)benzyl 2,4-dimethylbenzoate in CH₂Cl₂ was added methanesulfonic acid. The solution was stirred at room temperature for 2 hours. Then the solvents were evaporated and the compound was dried on the high vacuum to give mono-(methane sulfonic acid) salt of 4-(3-amino-1-(isoquinolin-6-ylamino)-1-oxopropan-2-yl)benzyl 2,4-dimethylbenzoate. A non-limiting example of a mono-(acid) salt prepared by this procedure is shown in Table 6.

TABLE 6

| | MW | Amount | mmol | equivalents | Yield |
|---|---|---|---|---|---|
| 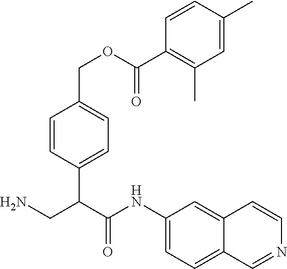 [SMILES: Cc1ccc(C(=O)OCc2ccc(C(CN)C(=O)Nc3ccc4cnccc4c3)cc2)c(C)c1] | 453.53 | 250 mg | 0.55 mmol | | |
| methanesulfonic acid | 96.11 | 35.8 μL | 0.55 mmol | 1.0 | 260 mg, 86% |

### Example 8. Synthesis of mono-(acid) salts of (S)-3-amino-2-(4-(hydroxymethyl)phenyl)-N-(isoquinolin-6-yl)propanamide

### General Procedure:

To a solution of (S)-3-amino-2-(4-(hydroxymethyl)phenyl)-N-(isoquinolin-6-yl)propanamide in EtOH at 0° C. was added a solution of H₂SO₄ in EtOH at 0° C. The solution was stirred for 10 min at 0° C. then warmed to room temperature for 5 min. The solvents were evaporated and the solids were dried on the high vacuum at 50° C. to give mono-(sulfuric acid) salt of (S)-3-amino-2-(4-(hydroxymethyl)phenyl)-N-(isoquinolin-6-yl)propanamide. A non-limiting example of a mono-(acid) salt prepared by this procedure is shown in Table 7.

TABLE 7

| | MW | Amount | mmol | equivalents | Yield |
|---|---|---|---|---|---|
| 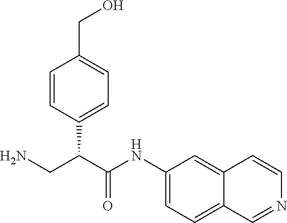 [SMILES: NC[C@@H](C(=O)Nc1ccc2cnccc2c1)c1ccc(CO)cc1] | 321.37 | 207 mg | 0.64 mmol | 1.0 | |
| sulfuric acid | 98.08 | 17.2 μL | 0.32 mmol | 0.5 | >95% |

### Example 9. Synthesis of mono-(acid) salts of (S)-3-amino-2-(4-(hydroxymethyl)phenyl)-N-(isoquinolin-6-yl)propanamide

### General Procedure:

To a solution of (S)-3-amino-2-(4-(hydroxymethyl)phenyl)-N-(isoquinolin-6-yl)propanamide in MeOH at 0° C. was added a solution of benzoic acid in MeOH at 0° C. The solution was warmed to room temperature and stirred for 20 minutes. The solvents were evaporated and the solids were dried on the high vacuum at 45° C. to give mono-(benzoic acid) salt of (S)-3-amino-2-(4-(hydroxymethyl)phenyl)-N-(isoquinolin-6-yl)propanamide. A non-limiting example of a mono-(acid) salt prepared by this procedure is shown in Table 8.

TABLE 8

| | MW | Amount | mmol | equivalents | MP |
|---|---|---|---|---|---|
| 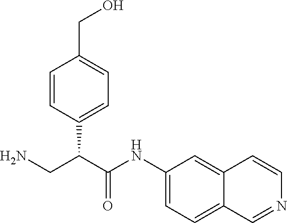 [SMILES: NC[C@@H](C(=O)Nc1ccc2cnccc2c1)c1ccc(CO)cc1] | 321.37 | 246 mg | 0.76 mmol | 1.0 | |
| benzoic acid | 122.12 | 93.3 mg | 0.76 mmol | 1.0 | 95-99° C. |

### Example 10. Synthesis of mono-(acid) salts of (S)-3-amino-2-(4-(hydroxymethyl)phenyl)-N-(isoquinolin-6-yl)propanamide

### General Procedure:

To a solution of (S)-3-amino-2-(4-(hydroxymethyl)phenyl)-N-(isoquinolin-6-yl)propanamide in EtOH at 0° C. was added benzenesulfonic acid and the solution was warmed to room temperature and stirred for 15 minutes. The solvents were evaporated and the solids were dried on the high vacuum at 45-50° C. to give mono-(benzenesulfonic acid) salt of (S)-3-amino-2-(4-(hydroxymethyl)phenyl)-N-(isoquinolin-6-yl)propanamide. A non-limiting example of a mono-(acid) salt prepared by this procedure is shown in Table 9.

TABLE 9

| | MW | Amount | mmol | equivalents | MP |
|---|---|---|---|---|---|
| 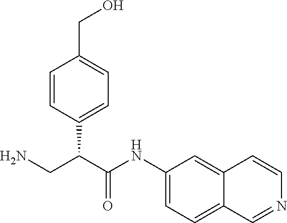 [SMILES: NC[C@@H](C(=O)Nc1ccc2cnccc2c1)c1ccc(CO)cc1] | 321.37 | 159 mg | 0.49 mmol | 1.0 | |
| benzenesulfonic acid | 158.18 | 78.1 mg | 0.49 mmol | 1.0 | 122-129° C. |

### Example 11. Synthesis of mono-(acid) salts of (S)-3-amino-2-(4-(hydroxymethyl)phenyl)-N-(isoquinolin-6-yl)propanamide

### General Procedure:

To a solution of (S)-3-amino-2-(4-(hydroxymethyl)phenyl)-N-(isoquinolin-6-yl)propanamide in EtOH at 0° C. was added p-toluenesulfonic acid monohydrate and the solution was warmed to room temperature and stirred for 15 minutes. The solvents were evaporated and the solids were dried on the high vacuum at 45-50° C. to give mono-(p-toluenesulfonic acid) salt of (S)-3-amino-2-(4-(hydroxymethyl)phenyl)-N-(isoquinolin-6-yl)propanamide. A non-limiting example of a mono-(acid) salt prepared by this procedure is shown in Table 10.

TABLE 10

| | MW | Amount | mmol | equivalents | MP |
|---|---|---|---|---|---|
| 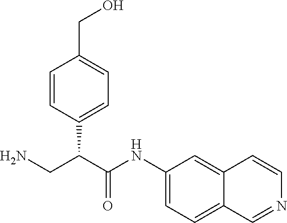 [SMILES: NC[C@@H](C(=O)Nc1ccc2cnccc2c1)c1ccc(CO)cc1] | 321.37 | 1.00 g | 3.1 mmol | 1.0 | |
| p-toluenesulfonic acid monohydrate | 190.22 | 596 mg | 3.1 mmol | 1.0 | 135-140° C. |

### Example 12. Synthesis of mono-(acid) salts of (R)-3-amino-2-(4-(hydroxymethyl)phenyl)-N-(isoquinolin-6-yl)propanamide

### General Procedure:

To a solution of (R)-3-amino-2-(4-(hydroxymethyl)phenyl)-N-(isoquinolin-6-yl)propanamide in EtOH at 0° C. was added p-toluenesulfonic acid monohydrate and the solution was warmed to room temperature and stirred for 15-20 minutes. The solvents were evaporated and the solids were dried on the high vacuum at 45-50° C. to give mono-(p-toluenesulfonic acid) salt of (R)-3-amino-2-(4-(hydroxymethyl)phenyl)-N-(isoquinolin-6-yl)propanamide. A non-limiting example of a mono-(acid) salt prepared by this procedure is shown in Table 11.

TABLE 11

| | MW | Amount | mmol | equivalents | Yield |
|---|---|---|---|---|---|
| 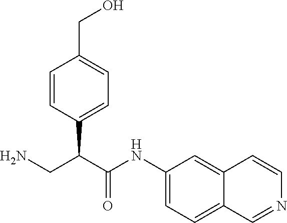 [SMILES: NC[C@H](C(=O)Nc1ccc2cnccc2c1)c1ccc(CO)cc1] | 321.37 | 1.58 g | 4.9 mmol | 1.0 | |
| p-toluenesulfonic acid monohydrate | 190.22 | 936 mg | 4.9 mmol | 1.0 | 2.1 g, 85% |

### Example 13. Synthesis of mono-(acid) salts of (S)-3-amino-2-(4-(hydroxymethyl)phenyl)-N-(isoquinolin-6-yl)propanamide

### General Procedure:

To a solution of (S)-3-amino-2-(4-(hydroxymethyl)phenyl)-N-(isoquinolin-6-yl)propanamide in EtOH at 0° C. was added methanesulfonic acid and the solution was warmed to room temperature and stirred for 20-30 minutes. The solvents were evaporated and the solids were dried on the high vacuum at 45-50° C. and transferred to a 1 dram vial to give mono-(methanesulfonic acid) salt of (S)-3-amino-2-(4-(hydroxymethyl)phenyl)-N-(isoquinolin-6-yl)propanamide. A non-limiting example of a mono-(acid) salt prepared by this procedure is shown in Table 12.

TABLE 12

| | MW | Amount | mmol | equivalents | MP |
|---|---|---|---|---|---|
| 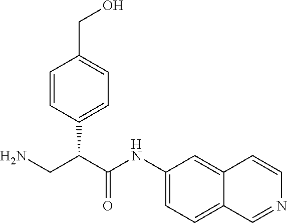 [SMILES: NC[C@@H](C(=O)Nc1ccc2cnccc2c1)c1ccc(CO)cc1] | 321.37 | 74.1 mg | 0.23 mmol | 1.0 | |
| methanesulfonic acid | 96.11 | 14.9 μL | 0.23 mmol | 1.0 | 90-100° C. |

### Example 14. Synthesis of mono-(acid) salts of (S)-3-amino-2-(4-(hydroxymethyl)phenyl)-N-(isoquinolin-6-yl)propanamide

### General Procedure:

To a solution of (S)-3-amino-2-(4-(hydroxymethyl)phenyl)-N-(isoquinolin-6-yl)propanamide in EtOH at 0° C. was added 2,4-dimethylbenzenesulfonic acid hydrate and the solution was warmed to room temperature and stirred for 20-30 minutes. The solvents were evaporated and the solids were dried on the high vacuum at 45-50° C. and transferred to a 1 dram vial to give mono-(2,4-dimethylbenzenesulfonic acid) salt of (S)-3-amino-2-(4-(hydroxymethyl)phenyl)-N-(isoquinolin-6-yl)propanamide. A non-limiting example of a mono-(acid) salt prepared by this procedure is shown in Table 13.

TABLE 13

| | MW | Amount | mmol | equivalents | MP |
|---|---|---|---|---|---|
| 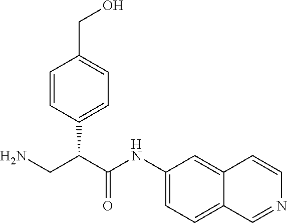 [SMILES: NC[C@@H](C(=O)Nc1ccc2cnccc2c1)c1ccc(CO)cc1] | 321.37 | 68.4 mg | 0.21 mmol | 1.0 | |
| 2,4-dimethyl-benzenesulfonic acid hydrate | 204.2 | 43.5 mg | 0.21 mmol | 1.0 | 108-115° C. |

### Example 15. Synthesis of mono-(acid) salts of (S)-3-amino-2-(4-(hydroxymethyl)phenyl)-N-(isoquinolin-6-yl)propanamide

### General Procedure:

To a solution of (S)-3-amino-2-(4-(hydroxymethyl)phenyl)-N-(isoquinolin-6-yl)propanamide in EtOH at 0° C. was added phenyl methanesulfonic acid and the solution was warmed to room temperature and stirred for 20-30 minutes. The solvents were evaporated and the solids were dried on the high vacuum at 45-50° C. and transferred to a 1 dram vial to give mono-(phenylmethanesulfonic acid) salt of (S)-3-amino-2-(4-(hydroxymethyl)phenyl)-N-(isoquinolin-6-yl)propanamide. A non-limiting example of a mono-(acid) salt prepared by this procedure is shown in Table 14.

TABLE 14

| | MW | Amount | mmol | equivalents | MP |
|---|---|---|---|---|---|
| 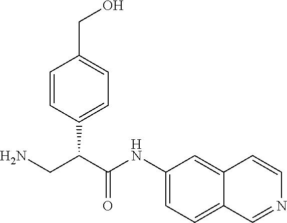 [SMILES: NC[C@@H](C(=O)Nc1ccc2cnccc2c1)c1ccc(CO)cc1] | 321.37 | 65.8 mg | 0.20 mmol | 1.0 | |
| phenyl methanesulfonic acid | 172.2 | 35.3 mg | 0.20 mmol | 1.0 | 86-92° C. |

### Example 16. Synthesis of mono-(acid) salts of (S)-3-amino-2-(4-(hydroxymethyl)phenyl)-N-(isoquinolin-6-yl)propanamide

### General Procedure:

To a solution of (S)-3-amino-2-(4-(hydroxymethyl)phenyl)-N-(isoquinolin-6-yl)propanamide in EtOH at 0° C. was added 4-chlorobenzenesulfonic acid hydrate and the solution was warmed to room temperature and stirred for 20-30 minutes. The solvents were evaporated and the solids were dried on the high vacuum at 45-50° C. and transferred to a 1 dram vial to give mono-(4-chlorobenzenesulfonic acid) salt of (S)-3-amino-2-(4-(hydroxymethyl)phenyl)-N-(isoquinolin-6-yl)propanamide. A non-limiting example of a mono-(acid) salt prepared by this procedure is shown in Table 15.

TABLE 15

| | MW | Amount | mmol | equivalents | MP |
|---|---|---|---|---|---|
| 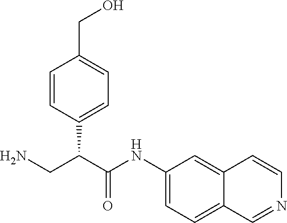 [SMILES: NC[C@@H](C(=O)Nc1ccc2cnccc2c1)c1ccc(CO)cc1] | 321.37 | 67 mg | 0.20 mmol | 1.0 | |
| 4-chlorobenzene-sulfonic acid hydrate | 210.6 | 43.8 mg | 0.20 mmol | 1.0 | 121-130° C. |

### Example 17. Synthesis of mono-(acid) salts of (S)-3-amino-2-(4-(hydroxymethyl)phenyl)-N-(isoquinolin-6-yl)propanamide

### General Procedure:

To a solution of (S)-3-amino-2-(4-(hydroxymethyl)phenyl)-N-(isoquinolin-6-yl)propanamide in EtOH at 0° C. was added [1,1′-biphenyl]-4-sulfonic acid and the solution was warmed to room temperature and stirred for 20-30 minutes. The solvents were evaporated and the solids were dried on the high vacuum at 45-50° C. and transferred to a 1dram vial to give mono-([1,1′-biphenyl]-4-sulfonic acid) salt of (S)-3-amino-2-(4-(hydroxymethyl)phenyl)-N-(isoquinolin-6-yl)propanamide. A non-limiting example of a mono-(acid) salt prepared by this procedure is shown in Table 16.

TABLE 16

| | MW | Amount | mmol | equivalents | MP |
|---|---|---|---|---|---|
| 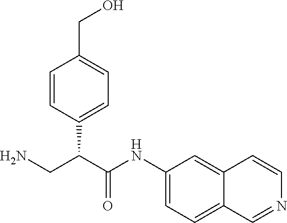 [SMILES: NC[C@@H](C(=O)Nc1ccc2cnccc2c1)c1ccc(CO)cc1] | 321.37 | 68 mg | 0.21 mmol | 1.0 | |
| [1,1′-biphenyl]-4-sulfonic acid | 234.27 | 49.8 mg | 0.21 mmol | 1.0 | 193-205 |

### Example 18. Synthesis of mono-(acid) salts of (S)-3-amino-2-(4-(hydroxymethyl)phenyl)-N-(isoquinolin-6-yl)propanamide

### General Procedure:

To a solution of (S)-3-amino-2-(4-(hydroxymethyl)phenyl)-N-(isoquinolin-6-yl)propanamide in EtOH at 0° C. was added cyclopentane sulfonic acid and the solution was warmed to room temperature and stirred for 20-30 minutes. The solvents were evaporated and the solids were dried on the high vacuum at 45-50° C. and transferred to a 1 dram vial to give mono(cyclopentane sulfonic acid) salt of (S)-3-amino-2-(4-(hydroxymethyl)phenyl)-N-(isoquinolin-6-yl)propanamide. A non-limiting example of a mono-(acid) salt prepared by this procedure is shown in Table 17.

TABLE 17

| | MW | Amount | mmol | equivalents | MP |
|---|---|---|---|---|---|
| 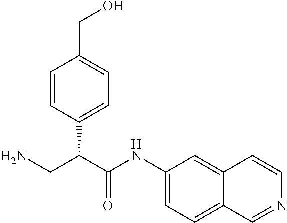 [SMILES: NC[C@@H](C(=O)Nc1ccc2cnccc2c1)c1ccc(CO)cc1] | 321.37 | 55 mg | 0.17 mmol | 1.0 | |
| Cyclopentane sulfonic acid | 150.2 | 26 mg | 0.17 mmol | 1.0 | 115-125° C. |

### Example 19. Synthesis of mono-(acid) salts of (S)-3-amino-N-(isoquinolin-6-yl)-2-(p-tolyl)propanamide (Scheme 4)

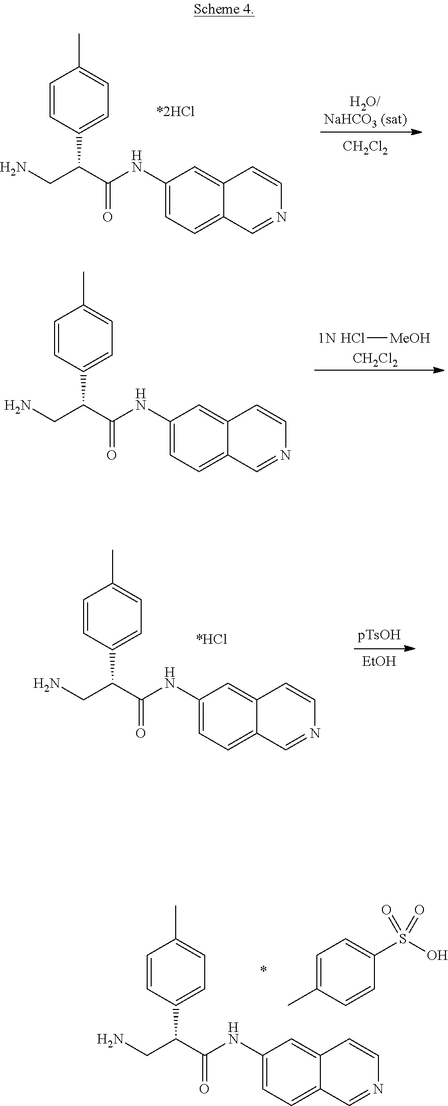

### Preparation of (S)-3-amino-N-(isoquinolin-6-yl)-2-(p-tolyl)propanamide

(hydrochloric acid) salt of (S)-3-amino-N-(isoquinolin-6-yl)-2-(p-tolyl)propanamide (1 g, 2.64 mmol) was dissolved in water (12 mL) and methylene chloride (10 mL) was added. Then NaHCO₃ (sat, 15 mL) was added to precipitate out. Extraction of the aqueous with CH₂Cl₂ and minimal MeOH (to dissolve the solids), followed by drying with Na₂SO₄, filtering and evaporation gave (S)-3-amino-N-(isoquinolin-6-yl)-2-(p-tolyl)propanamide (753 mg, 93%).

### Preparation of mono-(hydrochloric acid) salt of (S)-3-amino-N-(isoquinolin-6-yl)-2-(p-tolyl)propanamide

(S)-3-amino-N-(isoquinolin-6-yl)-2-(p-tolyl)propanamide (357 mg, 1.2 mmol) was dissolved in CH₂Cl₂ (5.6 mL) and cooled to 0° C. 1 N HCl-MeOH (1N, 1.2 mL, 1.2 mmol) was added to the reaction (dropwise quickly) and the solution stirred for 5 minutes at 0° C. then for 15 minutes at room temperature. The solvents were evaporated and the compound was dried on the high vacuum for approx. 14 hours at 45-50° C. to give mono-(hydrochloric acid) salt of (S)-3-amino-N-(isoquinolin-6-yl)-2-(p-tolyl)propanamide (368 mg g, 97%, mp=173-180° C.).

### Preparation of mono-(p-toluenesulfonic acid) salt of (S)-3-amino-N-(isoquinolin-6-yl)-2-(p-tolyl)propanamide

(S)-3-amino-N-(isoquinolin-6-yl)-2-(p-tolyl)propanamide (298 mg, 0.98 mmol) was dissolved in EtOH (4.7 mL) and cooled to 0° C. p-Toluenesulfonic acid monohydrate (186 mg, 0.98 mmol) was added to the reaction and the solution stirred for 25 minutes at room temperature. The solvents were evaporated and the compound was dried on the high vacuum for approx. 14 hours at 45-47° C. to give mono-(p-toluenesulfonic acid) salt of (S)-3-amino-N-(isoquinolin-6-yl)-2-(p-tolyl)propanamide (280 mg, 60%, mp=134-140° C.).

### Example 20. Synthesis of mono-(acid) salts of (S)-3-amino-2-(4-chlorophenyl)-N-(isoquinolin-6-yl)propanamide (Scheme 5)

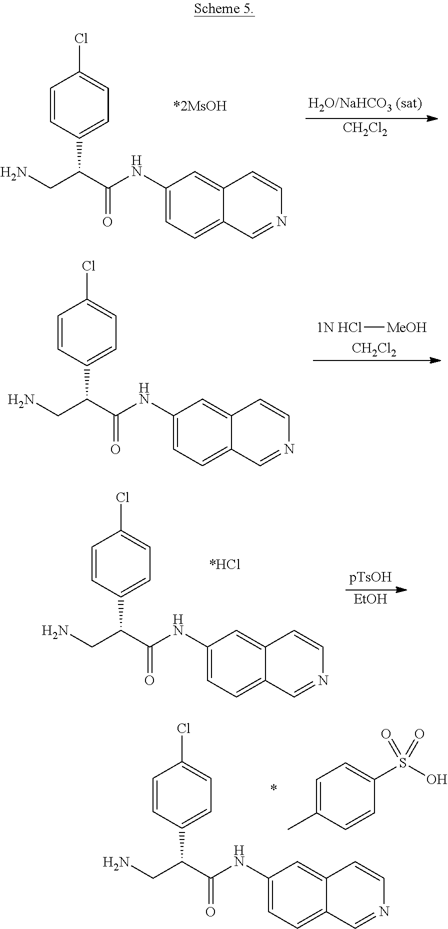

### Preparation of (S)-3-amino-2-(4-chlorophenyl)-N-(isoquinolin-6-yl)propanamide

di-(mesylic acid) salt of (S)-3-amino-2-(4-chlorophenyl)-N-(isoquinolin-6-yl)propanamide (1.6 g, 2.99 mmol) was dissolved in water (17 mL). Then NaHCO₃ (sat, 30 mL) was added to precipitate out. Extraction of the aqueous/precipates with CH₂Cl₂ and minimal MeOH (to dissolve the solids), followed by drying with Na₂SO₄, filtering and evaporation gave (S)-3-amino-2-(4-chlorophenyl)-N-(isoquinolin-6-yl)propanamide (885 mg, 91%).

### Preparation of mono-(hydrochloric acid) salt of (S)-3-amino-2-(4-chlorophenyl)-N-(isoquinolin-6-yl)propanamide

(S)-3-amino-2-(4-chlorophenyl)-N-(isoquinolin-6-yl)propanamide (347 mg, 1.1 mmol) was dissolved in CH₂Cl₂ (5.1 mL) and cooled to 0° C. 1 N HCl-MeOH (1N, 1.1 mL, 1.1 mmol) was added to the reaction (dropwise quickly) and the solution stirred for 5 minutes at 0° C. then for 15 minutes at room temperature. The solvents were evaporated and the compound was dried on the high vacuum for approx. 14 hours at 45-50° C. to give mono-(hydrochloric acid) salt of (S)-3-amino-2-(4-chlorophenyl)-N-(isoquinolin-6-yl)propanamide (351 mg g, 91%, mp=179-183° C.).

### Preparation of mono-(p-toluenesulfonic acid) salt of (S)-3-amino-2-(4-chlorophenyl)-N-(isoquinolin-6-yl)propanamide

(S)-3-amino-2-(4-chlorophenyl)-N-(isoquinolin-6-yl)propanamide (335 mg, 1.03 mmol) was dissolved in EtOH (5.0 mL) and cooled to 0° C. p-Toluenesulfonic acid monohydrate (196 mg, 1.03 mmol) was added to the reaction and the solution stirred for 15 minutes at room temperature. The solvents were evaporated and the compound was dried on the high vacuum for approx. 14 hours at 45-47° C. to give mono-(p-toluenesulfonic acid) salt of (S)-3-amino-2-(4-chlorophenyl)-N-(isoquinolin-6-yl)propanamide (466 mg, 91%, mp=195-200° C.).

### Example 21. Synthesis of mono-(acid) salts of (S)-4-(3-amino-1-(isoquinolin-6-ylamino)-1-oxopropan-2-yl)benzyl benzoate (Scheme 6)

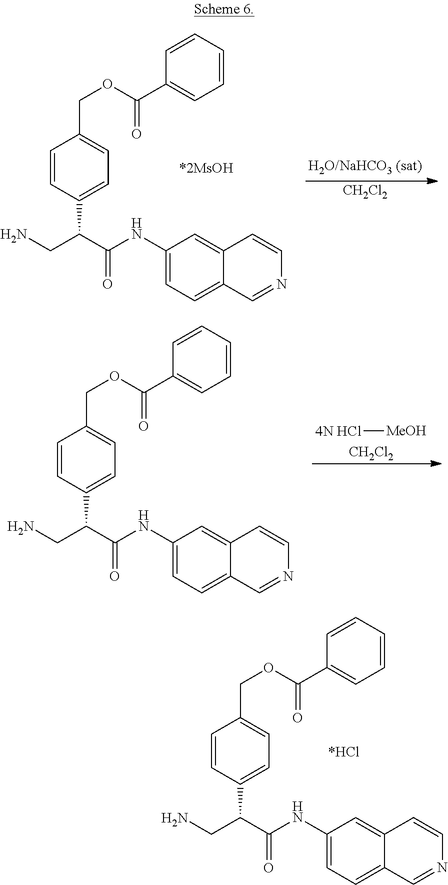

### Preparation of (S)-4-(3-amino-1-(isoquinolin-6-ylamino)-1-oxopropan-2-yl)benzyl benzoate

To the dihydrochloride salt of (S)-4-(3-amino-1-(isoquinolin-6-ylamino)-1-oxopropan-2-yl)benzyl benzoate (10.8 g, 21.8 mmol) was added a minimum amount of water and 1 N HCl (1-1.5 mL) to dissolve. Then NaHCO₃(sat) was added to precipitate out. The aqueous layer was extracted several times with CH₂Cl₂ and a little MeOH to recover (S)-4-(3-amino-1-(isoquinolin-6-ylamino)-1-oxopropan-2-yl)benzyl benzoate (8.4 g, 91%).

### Preparation of 1N HCl-MeOH

To MeOH (30 mL) cooled to 0° C. was added acetyl chloride (2.1 mL, 30 mmol) and the solution was stirred for 10 minutes at 0° C.

### Preparation of mono-(hydrochloric acid) salt of (S)-4-(3-amino-1-(isoquinolin-6-ylamino)-1-oxopropan-2-yl)benzyl benzoate

(S)-4-(3-amino-1-(isoquinolin-6-ylamino)-1-oxopropan-2-yl)benzyl benzoate (7.2 g, 16.9 mmol) was dissolved in CH₂Cl₂ (230 mL) and HCl-MeOH (1N, 17 mL, 17 mmol) was added to the reaction (dropwise quickly). The reaction mixture was stirred an additional 40 min. The solvents were evaporated and the compound was dried on the high vacuum for approx. 60 hours to give mono-(hydrochloric acid) salt of (S)-4-(3-amino-1-(isoquinolin-6-ylamino)-1-oxopropan-2-yl)benzyl benzoate (7.7 g, 98%). Recrystallization from MeOH and drying on the high vacuum gave the mono-(hydrochloric acid) salt of (S)-4-(3-amino-1-(isoquinolin-6-ylamino)-1-oxopropan-2-yl)benzyl benzoate (7 g, 89%, mp=235-239° C.).

1. A mono-(acid) salt of a compound of Formula (I): 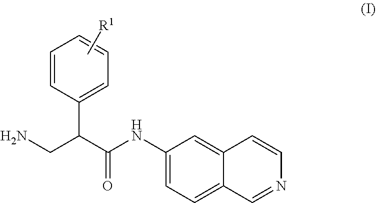
wherein
R¹ is H, halogen, —OH, —C₁₋₆ alkyl, —C₁₋₆ alkyl(R²)ₘ, —(CH₂)ₙOC(O)—(C₁₋₆ alkyl(R³)ₚ), or —(CH₂)ₙOC(O)—(C₆₋₁₀ aryl(R³)ₚ);
each R² is, independently, —OH or halogen;
each R³ is, independently, hydrogen, —OH, halogen, —C₁₋₆ alkyl, mono-halogen C₁₋₆ alkyl, di-halogen C₁₋₆ alkyl, or tri-halogen C₁₋₆ alkyl;
m is 1, 2, 3, 4, 5, or 6;
n is 1, 2, or 3; and
p is 0, 1, 2, 3, 4, 5, or 6.

2. The mono-(acid) salt of claim 1, wherein the acid is (−)-L-malic acid, (−)-L-pyroglutamic acid, (+)-camphor-10-sulfonic acid, (+)-camphoric acid, (+)-L-tartaric acid, 1-hydroxy-2-naphthoic acid, 2,2-dichloroacetic acid, 2-hydroxy-ethanesulfonic acid, 2-oxo-glutaric acid, 4-acetamido-benzoic acid, 4-amino-salicylic acid, acetic acid, adipic acid, alginic acid, benzenesulfonic acid, benzoic acid, capric acid, caproic acid, caprylic acid, carbonic acid, cinnamic acid, citric acid, cyclamic acid, D-glucoheptonic acid, D-gluconic acid, D-glucuronic acid, DL-lactic acid, DL-mandelic acid, dodecylsulfuric acid, ethane-1,2-disulfonic acid, ethanesulfonic acid, formic acid, fumaric acid, galactaric acid, gentisic acid, glutamic acid, glutaric acid, glycerophosphoric acid, glycolic acid, hippuric acid, hydrobromic acid, hydrochloric acid, isobutyric acid, lactobionic acid, L-ascorbic acid, L-aspartic acid, lauric acid, maleic acid, malonic acid, methanesulfonic acid, naphthalene-1,5-disulfonic acid, naphthalene-2-sulfonic acid, nicotinic acid, nitric acid, oleic acid, orotic acid, oxalic acid, palmitic acid, pamoic acid, phosphoric acid, propionic acid, p-toluenesulfonic acid, salicylic acid, sebacic acid, stearic acid, succinic acid, sulfuric acid, thiocyanic acid, or undecylenic acid.

3. The mono-(acid) salt of claim 1, wherein the acid is hydrochloric acid, methanesulfonic acid, p-toluenesulfonic acid, benzoic acid, sulfuric acid, acetic acid, phosphoric acid, citric acid, benzenesulfonic acid, sorbic acid, D-aspartic acid, L-aspartic acid, DL-aspartic acid, tartaric acid, fumaric acid, maleic acid, 2,4-dimethylbenzenesulfonic acid, phenylmethanesulfonic acid, 4-chlorobenzenesulfonic acid, [1,1′-biphenyl]-4-sulfonic acid, or cyclopentane sulfonic acid.

4-5. (canceled)

6. The mono-(acid) salt of claim 1, wherein the compound of Formula (I) is a compound of Formula (II):

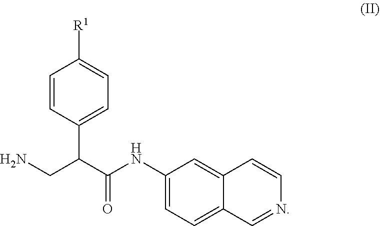

7-8. (canceled)

9. The mono-(acid) salt of claim 1, wherein the compound of Formula (I) is a compound of Formula (III):

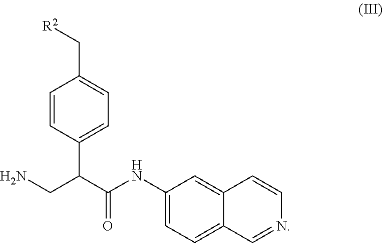

10-11. (canceled)

12. The mono-(acid) salt of claim 1, wherein the compound of Formula (I) is a compound of Formula (IV): 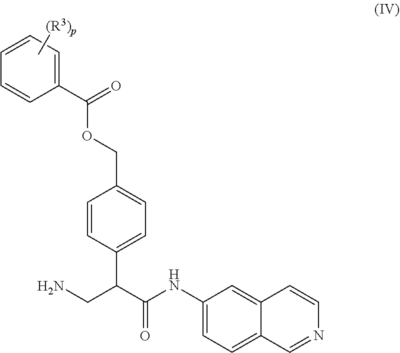
wherein
p is 0, 1, 2, 3, 4, or 5.

13-15. (canceled)

16. The mono-(acid) salt of claim 1, wherein p is 0, 1, 2, or 3.

17-18. (canceled)

18. The mono-(acid) salt of claim 1, wherein p is 2.

19. The mono-(acid) salt of claim 1, wherein R¹ is H, halogen, —OH, —C₁₋₆ alkyl, or —C₁₋₆ alkyl(R²)ₘ.

20. (canceled)

21. The mono-(acid) salt of claim 1, wherein R¹ is —(CH₂)ₙOC(O)—(C₆₋₁₀ aryl(R³)ₚ).

22. The mono-(acid) salt of claim 1, wherein R¹ is —(CH₂)ₙOC(O)-(phenyl(R³)ₚ).

23. The mono-(acid) salt of claim 1, wherein R² is —OH.

24. The mono-(acid) salt of claim 1, wherein
R¹ is —C₁₋₆ alkyl(R²)ₘ; and
R² is —OH.

25. The mono-(acid) salt of claim 1, wherein each R³ is, independently, —OH, halogen, or —C₁₋₆ alkyl.

26-27. (canceled)

28. The mono-(acid) salt of claim 1, wherein m is 1, 2, 3 or 4.

29. (canceled)

30. The mono-(acid) salt of claim 1, wherein n is 1 or 2.

31. (canceled)

32. The mono-(acid) salt of claim 1, wherein the compound of Formula (I) is:

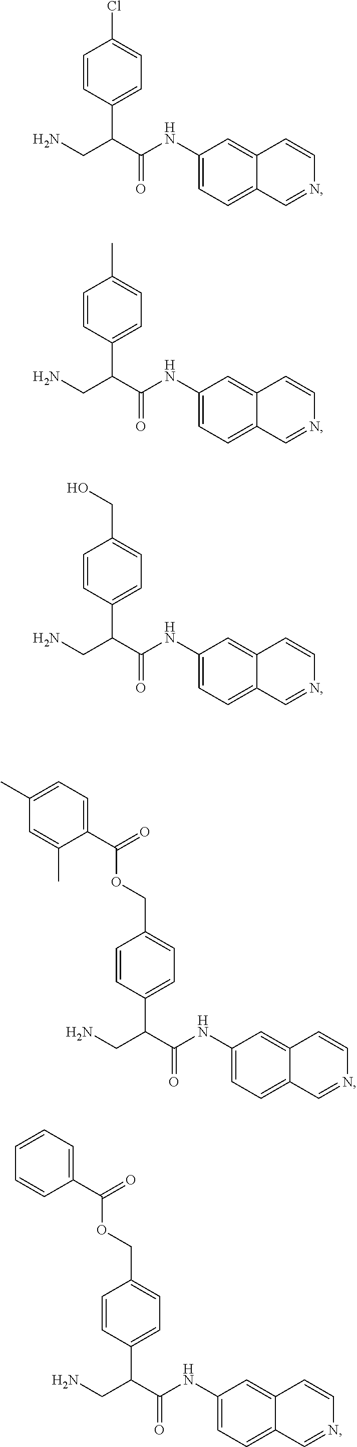 [SMILES: NCC(C(=O)Nc1ccc2cnccc2c1)c1ccc(Cl)cc1 ; Cc1ccc(C(CN)C(=O)Nc2ccc3cnccc3c2)cc1 ; NCC(C(=O)Nc1ccc2cnccc2c1)c1ccc(CO)cc1 ; Cc1ccc(C(=O)OCc2ccc(C(CN)C(=O)Nc3ccc4cnccc4c3)cc2)c(C)c1 ; NCC(C(=O)Nc1ccc2cnccc2c1)c1ccc(COC(=O)c2ccccc2)cc1]

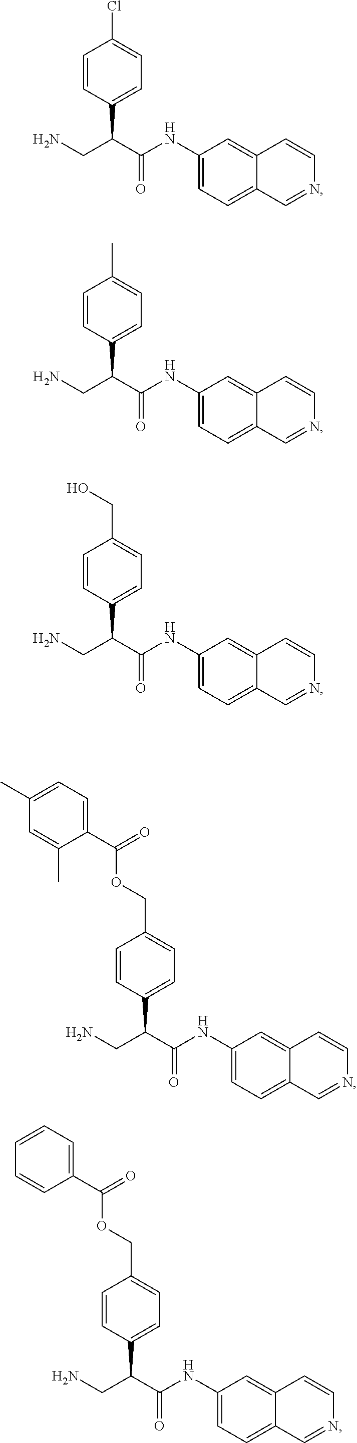 [SMILES: NC[C@H](C(=O)Nc1ccc2cnccc2c1)c1ccc(Cl)cc1 ; Cc1ccc([C@H](CN)C(=O)Nc2ccc3cnccc3c2)cc1 ; NC[C@H](C(=O)Nc1ccc2cnccc2c1)c1ccc(CO)cc1 ; Cc1ccc(C(=O)OCc2ccc([C@H](CN)C(=O)Nc3ccc4cnccc4c3)cc2)c(C)c1 ; NC[C@H](C(=O)Nc1ccc2cnccc2c1)c1ccc(COC(=O)c2ccccc2)cc1]

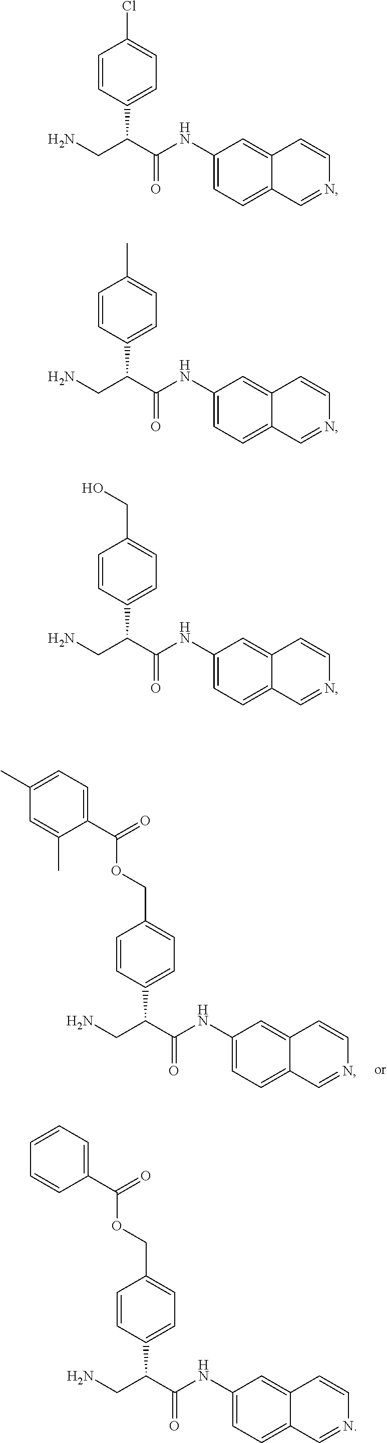 [SMILES: NC[C@@H](C(=O)Nc1ccc2cnccc2c1)c1ccc(Cl)cc1 ; Cc1ccc([C@@H](CN)C(=O)Nc2ccc3cnccc3c2)cc1 ; NC[C@@H](C(=O)Nc1ccc2cnccc2c1)c1ccc(CO)cc1 ; Cc1ccc(C(=O)OCc2ccc([C@@H](CN)C(=O)Nc3ccc4cnccc4c3)cc2)c(C)c1 ; "or" ; NC[C@@H](C(=O)Nc1ccc2cnccc2c1)c1ccc(COC(=O)c2ccccc2)cc1]

33. The mono-(acid) salt of claim 1, wherein the acid is p-toluenesulfonic acid, benzoic acid, benzenesulfonic acid, 2,4-dimethylbenzenesulfonic acid, phenylmethanesulfonic acid, 4-chlorobenzenesulfonic acid, [1,1′-biphenyl]-4-sulfonic acid, or cyclopentane sulfonic acid.

34. (canceled)

35. A composition, comprising the mono-(acid) salt of claim 1, or a combination thereof.

36. A composition comprising a combination of a mono-(acid) salt of Formula (Ia) and a mono-(acid) salt of Formula (Ib): 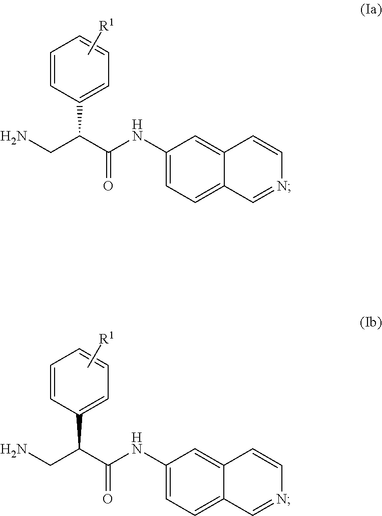
wherein
each R¹ is, independently, H, halogen, —OH, —C₁₋₆ alkyl, —C₁₋₆ alkyl(R²)ₘ, —(CH₂)ₙOC(O)—(C₁₋₆ alkyl(R³)ₚ), or —(CH₂)ₙOC(O)—(C₆₋₁₀ aryl(R³)ₚ);
each R² is, independently, —OH or halogen;
each R³ is, independently, hydrogen, —OH, halogen, —C₁₋₆ alkyl, mono-halogen C₁₋₆ alkyl, di-halogen C₁₋₆ alkyl, or tri-halogen C₁₋₆ alkyl;
m is, independently, 1, 2, 3, 4, 5, or 6;
n is, independently, 1, 2, or 3; and
p is, independently, 0, 1, 2, 3, 4, 5, or 6.

37. (canceled)

38. The composition of claim 35, wherein the composition comprises:
a combination of a mono-(acid) salt of (R)-3-amino-2-(4-(hydroxymethyl)phenyl)-N-(isoquinolin-6-yl)propanamide and a mono-(acid) salt of (S)-3-amino-2-(4-(hydroxymethyl)phenyl)-N-(isoquinolin-6-yl)propanamide;
a mono-(acid) salt of (rac)-3-amino-2-(4-(hydroxymethyl)phenyl)-N-(isoquinolin-6-yl)propanamide, (R)-3-amino-2-(4-(hydroxymethyl)phenyl)-N-(isoquinolin-6-yl)propanamide, or a mono-(acid) salt of (S)-3-amino-2-(4-(hydroxymethyl)phenyl)-N-(isoquinolin-6-yl)propanamide;
a combination of a mono-(acid) salt of (R)-3-amino-2-(4-chlorophenyl)-N-(isoquinolin-6-yl)propanamide and a mono-(acid) salt of (S)-3-amino-2-(4-chlorophenyl)-N-(isoquinolin-6-yl)propanamide;
a mono-(acid) salt of (rac)-3-amino-2-(4-chlorophenyl)-N-(isoquinolin-6-yl)propanamide, (R)-3-amino-2-(4-chlorophenyl)-N-(isoquinolin-6-yl)propanamide, or a mono-(acid) salt of (S)-3-amino-2-(4-chlorophenyl)-N-(isoquinolin-6-yl)propanamide;
a combination of a mono-(acid) salt of (R)-3-amino-N-(isoquinolin-6-yl)-2-(p-tolyl)propanamide and a mono-(acid) salt of (S)-3-amino-N-(isoquinolin-6-yl)-2-(p-tolyl)propanamide;
a mono-(acid) salt of (rac)-3-amino-N-(isoquinol in-6-yl)-2-(p-tolyl)propanamide, (R)-3-amino-N-(isoquinolin-6-yl)-2-(p-tolyl)propanamide, or a mono-(acid) salt of (S′)-3-amino-N-(isoquinolin-6-yl)-2-(p-tolyl)propanamide;
a combination of a mono-(acid) salt of (R)-4-(3-amino-1-(isoquinolin-6-ylamino)-1-oxopropan-2-yl)benzyl 2,4-dimethylbenzoate and a mono-(acid) salt of (S)-4-(3-amino-1-(isoquinolin-6-ylamino)-1-oxopropan-2-yl)benzyl 2,4-dimethylbenzoate;
a mono-(acid) salt of (rac)-4-(3-amino-1-(isoquinolin-6-ylamino)-1-oxopropan-2-yl)benzyl 2,4-di methyl benzoate, (R)-4-(3-amino-1-(isoquinolin-6-ylamino)-1-oxopropan-2-yl)benzyl 2,4-dimethylbenzoate, or a mono-(acid) salt of (S)-4-(3-amino-1-(isoquinolin-6-ylamino)-1-oxopropan-2-yl)benzyl 2,4-dimethylbenzoate;
a combination of a mono-(acid) salt of (R)-4-(3-amino-1-(isoquinolin-6-ylamino)-1-oxopropan-2-yl)benzyl benzoate and a mono-(acid) salt of (S)-4-(3-amino-1-(isoquinolin-6-ylamino)-1-oxopropan-2-yl)benzyl benzoate; or
a mono-(acid) salt of (rac)-4-(3-amino-1-(isoquinolin-6-ylamino)-1-oxopropan-2-yl)benzyl benzoate, (R)-4-(3-amino-1-(isoquinolin-6-ylamino)-1-oxopropan-2-yl)benzyl benzoate, or a mono-(acid) salt of (S)-4-(3-amino-1-(isoquinolin-6-ylamino)-1-oxopropan-2-yl)benzyl benzoate.

39-47. (canceled)

48. The composition of claim 35, wherein the composition is a pharmaceutical composition further comprising a pharmaceutically acceptable carrier.

49. A method of treating a disease in a subject in need thereof, comprising administering to the subject a therapeutically effective amount of the mono-(acid) salt of claim 1.

50. The method of claim 49, wherein the disease comprises glaucoma, an inflammatory eye disease, dry eye, ocular hypertension, or a neurodegenerative eye disease.

51. The method of claim 49, wherein the disease comprises diabetic eye disease, wet age-related macular degeneration, or dry age-related macular degeneration.

52-54. (canceled)

53. The method of claim 49, wherein the administering to the subject is administering topically to an eye of the subject.

54. The method of claim 49, wherein the administering to the subject is administering topically to an eyelid of the subject.

55. A method of reducing intraocular pressure in a subject in need thereof, comprising administering to the subject a therapeutically effective amount of the mono-(acid) salt of claim 1.

56. The method of claim 55, wherein the administering to the subject is administering topically to an eye of the subject.

57. The method of claim 55, wherein the administering to the subject is administering topically to an eyelid of the subject.

58. A method of modulating kinase activity in a cell, comprising contacting the cell with an amount of the mono-(acid) salt of claim 1 effective to inhibit the kinase.

59. The method of claim 58, wherein the cell is in a subject.

60. The method of claim 59, wherein the subject is a human subject.

61. A kit comprising the mono-(acid) salt of one of claims 1-14 and instructions for use thereof.

62. An article of manufacture comprising the mono-(acid) salt of claim 1, and instructions for use thereof.

63. A mono-(hydrochloric acid) salt of (S)-4-(3-amino-1-(isoquinolin-6-ylamino)-1-oxopropan-2-yl)benzyl 2,4-dimethylbenzoate, a monohydrate of a mono-(hydrochloric acid) salt of (S)-4-(3-amino-1-(isoquinolin-6-ylamino)-1-oxopropan-2-yl)benzyl 2,4-dimethylbenzoate, or a hemihydrate of a mono-(hydrochloric acid) salt of (S)-4-(3-amino-1-(isoquinolin-6-ylamino)-1-oxopropan-2-yl)benzyl 2,4-dimethylbenzoate.

64-65. (canceled)
